(19) United States
(12) Patent Application Publication
(10) Pub. No.: US 2022/0054540 A1
(43) Pub. Date: Feb. 24, 2022

(54) Ferric Carboxymaltose In The Treatment Of Heart Failure And In The Treatment Of Congestion In Heart Failure

(71) Applicant: Vifor (International) AG, St. Gallen (CH)

(72) Inventors: Claudio Sergio Mori, Seuzach (CN); Darlington Obinnaya Okonko, London (GB)

(21) Appl. No.: 17/416,695

(22) PCT Filed: Dec. 20, 2019
(86) PCT No.: PCT/EP2019/086612

§ 371 Date: Jun. 21, 2021

(30) Foreign Application Priority Data
Dec. 21, 2018 (EP) 18215138.1

(51) Int. Cl. A61K 33/26; A61K 9/00; A61P 9/04

(52) U.S. Cl. CPC A61K 33/26; A61P 9/04; A61K 9/0019

(57) ABSTRACT

The invention relates to intravenously administered ferric carboxymaltose for the use as a diuretic medicament, in particular for the treatment of congestion, or of congestion associated with impaired function of the heart and/or associated with deterioration of the function of the heart in a patient suffering from heart failure with iron deficiency or in a patient being iron deficient and at risk of developing heart failure.

The present invention relates to an intravenously administered ferric carboxymaltose for the use as a diuretic medicament, in particular for the treatment of congestion, or of congestion associated with impaired function of the heart and/or associated with deterioration of the function of the heart in a patient suffering from heart failure with iron deficiency or in a patient being iron deficient and at risk of developing heart failure. The invention further relates to a method of treatment of congestion, or of congestion associated with impaired function of the heart and/or associated with deterioration of the function of the heart in a patient suffering from heart failure with iron deficiency or in a patient being iron deficient and at risk of developing heart failure, wherein an intravenously administered ferric carboxymaltose is administered to said patient.

Heart failure (HF) is a complex clinical syndrome affecting approximately 1-2% of the adult population in developed countries, rising to 10% or more among people over 70 years of age.

It results from a state where the heart is unable to provide the absolute or relative blood volume that meets the oxygen demand of the peripheral organs. As the condition progresses and systolic and/or diastolic heart function eventually deteriorates, this insufficiency increases.

The general indication “heart failure” can be further divided into several sub-classes, depending on the underlying cause, symptoms and stage of severity.

In one aspect, HF can be subdivided into systolic and diastolic HF. In systolic HF, the left ventricle (LV), which pumps out oxygenated blood to the body, is either unable to contract normally or sufficiently resulting in reduced pumping action and consequently an ejection fraction (EF)<50% of the normal volume of blood expelled with each beat. This is referred to as HF with reduced EF, HFrEF. Alternatively, in diastolic HF, the heart cannot relax properly in the resting period between two heart beats resulting in reduced filling of the left and/or right ventricle(s). This is termed diastolic HF or dysfunction or HF with preserved ejection fraction (HFpEF). Diastolic HF often occurs as a result of systolic HF through ventricular interdependence or when LV failure leads to elevated left-sided fluid pressure, which is transferred back to the lungs thereby increasing pulmonary blood pressures. Increased pulmonary blood pressures affect the right ventricular afterload. This afterload (pulmonary venous hypertension extends to pulmonary arteries) leads to dysfunction of the right ventricle (RV), tricuspid regurgitation and subsequent further RV impairment. The right ventricle (RV), which normally pumps deoxygenated blood back to the lungs via the right atrium, loses its ability to pump. As a result blood backs up in the body's veins leading to systemic congestion (edemal swelling of the legs, ankles, and abdomen, pulmonary edema and pleural effusions, hepatic congestion). This particular stage or severity of heart failure is also called “congestive heart failure”, which constitutes as specific subgroup or manifestation of “heart failure”, as described in several definitions. For example, the health information portal Healthline.com provides the following information (https://www.healthline.com/health/congestive-heart-failure):

Congestive heart failure (CHF) is a chronic progressive condition that affects the pumping power of the heart muscles. While often referred to simply as “heart failure”, CHF specifically refers to the stage in which fluid builds up around the heart and causes it to pump inefficiently.

Further, Martens P and Mullens W: “How to tackle congestion in acute heart failure”; Korean J Intern Med. 2018, 33:462-473 describe congestion as follows:
“Congestion is defined as the signs and symptoms of extracellular fluid accumulation, instigated by an increase in left-sided cardiac filling pressure [14]. This definition recognizes that poor cardiac function is a prerequisite for developing congestion. However, numerous other organs play a role in the development of congestion, including the splanchnic veins and the interstitial and endothelial tissues. The use of implantable hemodynamic monitoring devices have significantly contributed to our understanding of how CHF develops into AHF [15].”

If the symptoms and signs of HF last for a certain period of time, the condition can be defined as chronic HF. The term stable HF is used only when the symptoms of HF are well-controlled for at least a month. The worsening of chronic stable HF is termed decompensated HF, while a sudden aggravation or failure leading to hospitalization is termed acute HF. The initial development of HF symptoms due to illnesses such as myocardial infarction or myocarditis is referred to as de novo HF. The term compensatory HF is used in HF patients who are asymptomatic or show an improvement in their condition for a certain period of time. Refractory HF is advanced HF, i.e. patients display advanced structural heart disease and marked symptoms of heart failure at rest despite dietary modification, salt restriction and maximal medical therapy including ACE inhibitors, angiotensin II receptor blockers, digitalis, diuretics and beta blockers. The term congestive HF, mainly used in the United States, indicates acute and chronic HF with signs of salt or fluid retention.

A symptomatic classification system used to determine the severity of the condition is the New York Health Association (NYHA) Functional Classification System with classes I to IV according to the signs and symptoms displayed by the heat failure patient as shown in Table 1.

TABLE 1

| Class | Description |
|---|---|
| Class I | Patients have cardiac disease but without the |
| | resulting limitations of physical activity. |
| | Ordinary physical activity does not cause undue |
| | fatigue, palpitation, dyspnoea, or anginal pain. |
| Class II | Patients have cardiac disease resulting in slight |
| | limitation of physical activity. They are comfortable |
| | at rest. Ordinary physical activity results in fatigue, |
| | palpitation dyspnoea, or anginal pain. |
| Class III | Patients have cardiac disease resulting in marked |
| | limitation of physical activity. They are comfortable |
| | at rest. Less than ordinary physical activity causes |
| | fatigue, palpitation, dyspnoea, or anginal pain. |
| Class IV | Patients have cardiac disease resulting in inability |
| | to carry on any physical activity without discomfort. |
| | Symptoms of cardiac insufficiency or of the anginal |
| | syndrome may be present even at rest. If any physical |
| | activity is undertaken, discomfort is increased. |

The American College of Cardiology/American Heart Association (ACC/AHA) heart failure classification system with stages A to D is based on the cardiac remodeling observed in heart failure, i.e. the structural changes of the heart and is summarized below.

Stage A: Patients at risk for heart failure who have not yet developed structural heart changes i.e. patients with coronary artery disease without prior myocardial infarction, hypertension, or diabetes mellitus without impaired left ventricular (LV) function, LV hypertrophy (LVH), or geometric chamber distortion. There is no corresponding NYHA functional class.

Stage B: Patients with structural heart disease who have not yet developed symptoms of heart failure, i.e. patients who are asymptomatic but who have reduced ejection fraction, left ventricular hypertrophy (LVH) and/or impaired LV function, chamber enlargement. This stage corresponds to patients with NYHA class I.

Stage C: Patients who have developed structural heart disease with current or past symptoms of clinical heart failure, i.e. shortness of breath and fatigue, reduced exercise tolerance

This stage corresponds with NYHA classes I, II, III and IV.

Stage D: Patients with refractory heart failure requiring advanced intervention, i.e. patients who have marked symptoms at rest despite maximal medical therapy. Patients at this stage may be eligible to receive mechanical circulatory support such as biventricular pacemakers or a left ventricular assist device, receive continuous inotropic infusions, undergo procedures to facilitate fluid removal, or undergo heart transplantation or other procedures. This stage corresponds with NYHA class IV.

The ACC/AHA classification differs from the NYHA system in that the NYHA system classifies patients based on symptoms alone and allows for patients to move back and forth between the individual classes. According to the ACC/AHA classification, once a patient progresses within the spectrum of A to D, no return to a previous stage is possible.

There are several different underlying causes of heart failure. Often, a myocardial abnormality causing systolic and/or diastolic ventricular dysfunction will exist. However, abnormalities of the valves, pericardium, endocardium, heart rhythm and conduction may also cause heart failure. Patients carrying a high risk of developing heart failure are those suffering from coronary artery disease, having high blood pressure, pulmonary arterial hypertension, abnormal heart valves, heart muscle disease such as dilated or hypertrophic cardiomyopathy, myocarditis, congenital heart disease, severe lung disease, diabetes, obesity, sleep apnea and patients that have previously suffered from myocardial infarctions. Other conditions that carry a risk, albeit less frequently, of leading to temporary heart failure are a low count of red blood cells (anemia), hyperparathyroidism and abnormal heart rhythm such as arrhythmia or dysrhythmia.

An overview of the various underlying causes and symptoms of the general disease “heart failure” is also given on a further health information portal NHS.uk (https://www.nhs.uk/conditions/heart-failure), which also shows the complexity of the disease and its various manifestations.

Symptoms and signs of HF are often unspecific and sometimes hard to detect. While symptoms and signs are helpful in the assessment of progression of HF, in particular when congestion is monitored, they cannot be relied upon in isolation with respect to diagnosis of the condition. A number of methods are available to diagnose heart failure more reliably. Therefore, patients suspected of suffering from HF are subjected to a chest X-ray to check for an enlarged heart and pulmonary venous congestion (edema) as well as blood tests including complete blood cell count (CBC), measurements of serum electrolyte levels, assessment of renal and hepatic function and 12-lead electrocardiography (ECG). If the results obtained are indicative of heart failure, biomarkers such as the plasma concentration of brain natriuretic peptide (BNP) and its precursor the N-terminal brain natriuretic peptide (NT-proBNP) are determined with BNP 35 pg/mL and NT-proBNP 125 pg/mL in a non-acute setting and BNP 100 pg/mL and NT-proBNP 300 pg/mL indicating HF, respectively. Further investigations involve transthoracic echocardiography, which provides detailed information on the heart's function and structure, such as immediate information on chamber volumes, ventricular systolic and diastolic function, wall thickness, valve function and pulmonary hypertension. In some cases cardiac MRI and CT imaging are used to further determine the extent and possible cause of HF.

Typical symptoms and signs of heart failure, although non-specific, are dyspnea, orthopnea, paroxysmal nocturnal dyspnea, tachycardia fatigue, increased time to recovery after exercise, chronic coughing or wheezing, irregular pulse, tachypnea (>16 breaths/min), Cheyne Stokes respiration, hepatomegaly, cold extremities, oliguria, narrow pulse pressure, ascites and cachexia, nausea, loss of appetite, weight loss (in advanced HF), satiety, depression, weight gain (2+kg/week), palpitation, paroxysmal nocturnal dyspnea, exercise intolerance, delayed recovery from exercise, dizziness, bloated feeling, nocturnal cough, bendopnea, syncope, confusion (especially in the elderly) or impaired thinking and may be accompanied by signs such as hepatojugular reflux, third heart (S3) sound, laterally displaced apical impulse, cardiac murmur, reduced air entry and dullness to percussion at lung base (pleural effusion), peripheral edema (ankle, sacral, scrotal), pulmonary crackles, rales and elevated jugular venous pressure caused by structural and/or functional cardiac abnormality, which results in reduced cardiac output and/or elevated intracardiac pressures at rest or during stress.

In a manifestation of particular interest for the present invention a symptom observed both in chronic as well as acute heart failure patients is congestion also known as fluid retention or fluid overload. Congestion can be divided into two general categories, i.e. hemodynamic congestion and clinical congestion. Hemodynamic congestion refers to the state of increased intra-cardiac filling pressures caused by volume overload, i.e. high left ventricular diastolic pressure (LVDP) accompanied by cardiopulmonary volume overload that can occur in the absence of clinically evident signs or symptoms. Clinical congestion refers to the presence of signs and symptoms related to elevated intra-cardiac filling pressures and manifests itself through breathlessness, rales and crackles due to pulmonary congestion and pleural effusions as well as ascites due to hepatic congestion and ankle swelling due to peripheral edema.

Congestion can be assessed by a clinical congestion scale based on lung auscultation (normal, presence of basal mid-zone or diffuse crepitations), jugular venous pressure (JVP) (not visible, raised 1-4, raised to earlobe), peripheral oedema (none, ankles, below or above knees) and liver examination (not palpable, palpable) with one point attributed for each degree of severity and a total possible score of nine as well as by echocardiographic imaging of the inferior vena cava diameter (not congested IVC ≤16 mm; mildly congested: IVC 17-20 mm; severely congested ≥21 mm).

As blood backs up in the body's veins due to LV, RV or biventricular impairment or dysfunction, this leads to edemal swelling of the legs, ankles, and abdomen, pulmonary edema and pleural effusions. As edema worsens, the patient suffers from respiratory distress as well as impaired hepatic function. Further, the kidneys are affected by congestion due to an inability to eliminate sodium and excess water brought about by a transrenal pressure gradient. Renal water and sodium retention, in turn, give rise to intravascular and interstitial fluid volume expansion and redistribution. The kidney acts as an early responder to the myocardial dysfunction and resulting arterial underfilling with reduction in effective circulating blood volume (BV). While initial sympathetic-driven vasoconstriction maintains organ perfusion-pressure in the short term, interstitial volume expansion occurs gradually to compensate for increased intravascular plasma volume (PV), giving rise to increased interstitial tissue pressure. Considering that only 30 to 40% of total BV normally resides in the arterial circulation and even less in systolic HF, a marked tissue expansion is to be expected. The effects of anemia, a prevalent co-morbidity of HF, can be exacerbated by dilution caused by congestion, which also further impacts the signs and symptoms of HF.

It is known that clinical, echocardiographic and biochemical evidence of congestion in heart failure is associated with an increased rate of hospitalization and higher mortality. In particular, this is true for acute heart failure syndrome (AHFS), where congestion is an important predictor of both mortality and morbidity. As a result, congestion is an essential evaluative and therapeutic target in AHFS patients. It is plausible that early identification of hemodynamic congestion, before the clinical manifestations are present, could reduce the need for hospital admission and readmission. It is also known that congestion in heart failure drives dyspnea and exercise intolerance. Further, given the fact that not only the heart but also the liver and the kidneys are affected, specifically treating congestion in heart failure is therefore of great importance.

Up until now, the standard of care in the treatment of congestion in heart failure has been the administration of conventional diuretics, in particular loop (high-ceiling) diuretics acting on the Loop of Henle such as furosemide (loop diuretics), as explained in P. Pellicori et al., Cardiac Dysfunction, Congestion and Loop Diuretics: their Relationship to Prognosis in Heart Failure, Cardiovasc. Drugs Ther. (2016), 30, 599-609. This conventional diuretic treatment relieves symptoms and signs of congestion as these medications facilitate the elimination of fluid from the body. However, it has been observed that patients with heart failure with a left ventricular ejection fraction (LVEF) below and above 50% and evidence of congestion who receive higher doses of loop diuretics have a worse one-year outcome and a greater risk of adverse events than heart failure patients not taking loop diuretics. Also, while conventional diuretics have been observed to alleviate the signs and symptoms associated with congestion, no evidence has been gathered to point to an effect of conventional diuretics on slowing or preventing heart failure progression. Rather, it is thought that administration of conventional diuretics activates the renin-angiotensin-aldosterone and sympathetic nervous systems thereby contributing to the progression and adverse outcome of heart failure.

There is thus a need to overcome the drawbacks of conventional therapy with conventional diuretics and to provide a treatment that addresses congestion and its signs and symptoms while influencing disease progression of heart failure in a positive way.

### DESCRIPTION OF THE INVENTION

The inventors of the present invention surprisingly found the suitability of ferric carboxymaltose to act as a diuretic, in particular in patients suffering from heart failure with iron deficiency or in patients being iron deficient and at risk of developing heart failure, when administered intravenously. It surprisingly turned out that ferric carboxymaltose can be used to effectively treat or prevent congestion in patients suffering from heart failure with iron deficiency or in patients being iron deficient and at risk of developing heart failure.

Thus, a first aspect of the invention relates to intravenously administered ferric carboxymaltose for the use as a diuretic, in particular in patients suffering from heart failure with iron deficiency or in patients being iron deficient and at risk of developing heart failure.

In a more particular aspect, the invention relates to intravenously administered ferric carboxymaltose for the use in the treatment of congestion in patients suffering from heart failure with iron deficiency or in patients being iron deficient and at risk of developing heart failure.

In an even more particular aspect, the invention relates to intravenously administered ferric carboxymaltose for the use in the treatment of congestion associated with impaired function of the heart or associated with deterioration of the function of the heart in patients suffering from heart failure with iron deficiency or in patients being iron deficient and at risk of developing heart failure.

Depending on the severity, ferric carboxymaltose can be used alone or in a combination therapy with the above mentioned conventional diuretic medicaments.

Congestion is synonymous with fluid retention and fluid or volume overload and manifests itself as systemic congestion, i.e. peripheral edema, pulmonary edema, pleural effusions or, at a more advanced stage, as hepatic congestion and ascites, which is an accumulation of fluid, usually serous fluid which is a pale yellow and clear fluid, that accumulates in the abdominal (peritoneal) cavity. Typical signs of congestion are ankle swelling and pulmonary rales and crackling, breathlessness as well as daily weight changes of more than two to three pounds in 24 hours.

The treatment of congestion in a patient suffering from heart failure with iron deficiency or in a patient being iron deficient and at risk of developing heart failure comprises the improvement of congestion, the delay of onset of congestion, the delay of recurrence of congestion, the delay of deterioration of congestion, the prevention of onset of congestion, the prevention of recurrence of congestion and/or the prevention of deterioration of congestion.

A patient at risk of developing HF is a patient that has not yet developed structural heart changes indicative of heart failure i.e. a patient with coronary artery disease with or without prior myocardial infarction, hypertension, pulmonary arterial hypertension, abnormal heart valves, heart muscle disease, myocarditis, congenital heart disease, severe lung disease, diabetes mellitus, obesity, sleep apnea without impaired left ventricular (LV) function, LV hypertrophy (LVH), or geometric chamber distortion. Further, patients having a low count of red blood cells (anemia), hyperparathyroidism and abnormal heart rhythm such as arrhythmia or dysrhythmia all carry an increased risk of developing HF.

Iron deficiency is a well-known and widespread co-morbidity in heart failure patients suffering either from chronic or acute heart failure. Iron deficiency (ID) is defined by a serum ferritin <150 ug/L or ferritin between 150 to 249 ug/L with a transferrin saturation (TSAT)<30%. HF patients with iron deficiency anemia (IDA) have in addition to the above laboratory values hemoglobin (Hb) levels between 9.5 and 13.5 g/dl. It is important to note, is that ID can be present in the absence of IDA, i.e. it can be considered a separate medical condition.

It has previously been shown that an intravenously administered iron delivery product, specifically ferric carboxymaltose, when administered to patients suffering from heart failure and iron deficiency with or without anemia, leads to an improvement or a stabilization of certain specific aspects of chronic heart failure, i.e. very specific symptoms associated with chronic heart failure. (Anker S D, Comin Colet J, et al. Ferric carboxymaltose in patients with heart failure and iron deficiency. N Engl J Med. 2009; 361:1-13 and Anker S D, Colet J C et al.; FAIR-HF committees and investigators. Rationale and design of Ferinject assessment in patients with Iron deficiency and chronic Heart Failure (FAIR-HF) study: a randomized, placebo-controlled study of intravenous iron supplementation in patients with and without anaemia. Eur J Heart Fail. 2009 November; 11(11):1084-91. Anker S D et al., “Effects of ferric carboxymaltose on hospitalizations and mortality rates in iron-deficient heart failure patients: an individual patient data meta-analysis”, 2018. Ponikowski P et al., “Beneficial effects of long-term intravenous iron therapy with ferric carboxymaltose in patients with symptomatic heart failure and iron deficiency”, 2014)

Rustam I et al., “Intravenous iron without erythropoietin for the treatment of iron deficiency anemia in patients with moderate to severe congestive heart failure and chronic kidney insufficiency”, 2008 described the administration of iron sucrose to patients with moderate to severe congestive heart failure and anemia but provide no teaching about any advantageous effects of ferric carboxymaltose as a diuretic in the treatment of congestion.

A publication of the applicant Vifor Pharma Ltd.: “Vifor Pharma announces three outcomes trials in heart failure and deficiency”, 2017 provides an overview of clinical trials run by Vifor Pharma but also remains silent about a possible efficacy of ferric carboxymaltose as a diuretic in the treatment of congestion.

So, up until now, no studies have been conducted investigating the impact of an iron therapy with ferric carboxymaltose on congestion in heart failure patients and none have applied reliable metrics of fluid overload to demonstrate this relationship.

It has now been demonstrated for the first time that intravenously administered ferric carboxymaltose can be administered to heart failure patients with iron deficiency in order to have a direct positive effect on congestion, in particular on congestion associated with impaired function of the heart and associated with deterioration of the function of the heart.

This is all the more surprising as many publications concerning ferric carboxymaltose report clinical congestion in the form of peripheral edema as one of the most commonly occurring adverse events related to the treatment of patients with ferric carboxymaltose (e. g. Wajeh Y. Qunibi et al., Nephrol. Dial. Transplant (2011), 26, 1599-1607).

Using weight recordings and hematocrit (HCT or Ht) values of a sub-set of participants of the previously conducted and published FAIR-HF trial ((Ferinject® Assessment in patients with Iron deficiency and chronic Heart Failure); Anker S D, Comin Colet J, et al. Ferric carboxymaltose in patients with heart failure and iron deficiency. N Engl J Med. 2009; 361:1-13), the applicant was able to calculate actual and ideal plasma volumes (PV). The actual and ideal PV values obtained were then used to calculate the relative plasma volume status (PVS), a novel index of congestion indicating the degree to which patients have deviated from their ideal PV.

Plasma volume (PV) expansion underlies systemic congestion in chronic heart failure, and can be objectively estimated using validated equations based on weight and hematocrit (Hct) (Hakim R M. “Plasmapheresis” in Handbook of dialysis, third edition, Daugirdas J T, Blake P G, Ing T S, Eds. Lippincott, Williams and Wilkins, 2001; 236 and Longo D. “Table 218: Body fluids and other mass data” in Harrison's Principles of Internal Medicine, 18th ed, Longo D L, Fauci A S, Kasper D L, Hauser S L, Jameson J L, Loscalzo J, Eds. McGraw-Hill, 2011, pp. A-1). A recent analysis showed that calculated PV values significantly mirror those measured using gold standard radioisotope assays. Moreover, in a trial dataset of 5002 CHF patients, relative PVS predicted outcomes even after adjustment for 22 variables including natriuretic peptides (Ling H Z, Flint J, et al. Calculated plasma volume status and prognosis in chronic heart failure. Eur J Heart Fail 2015; 17:35-43.).

The applicant sought to determine the distribution, correlates, and prognostic utility of calculated PVS in chronic heart failure patients enrolled in the large FAIR-HF trial. Moreover, the positive impact of iron therapy with ferric carboxymaltose (FCM) on calculated relative PVS and clinical congestion was investigated in this cohort.

The findings of the study show that compared to placebo, intravenous iron therapy with FCM is associated with greater reductions in relative PVS and other measures of clinical congestion such as weight and peripheral edema. The impact of FCM on relative PVS and weight is already evident after four weeks following FCM administration supporting a direct decongestive effect of iron repletion in CHF. The reductions in relative PVS at four weeks and the fact that this is paralleled by attenuations in weight, which is regarded as the best clinical barometer of clinical congestion, suggests that FCM has a true and early decongestive effect. Treatment with FCM is considered to be linked to attenuations in relative PVS for a number of reasons. First, it could be argued that decrements in relative PVS merely reflect the increments in Hct seen with FCM and are not truly indicative of a change in clinical congestion. However, FCM treatment has also been shown to lead to reductions in weight. Given that weight is clinically accepted to be the best gauge of short-term PV changes, and that no evidence exists to suggest that FCM triggers non-edematous weight loss, it is assumed that FCM truly diminishes clinical congestion.

Further results of the study show an increase in relative PVS over time being associated with worsening symptoms and exercise capacity irrespective of treatment allocation and that higher relative PVS values predicted an increased risk of death or hospitalization over the coming six months. Moreover, the study demonstrates that plasma volume status, as estimated by the relative PVS equation, is mildly contracted in the stable population investigated and relates to age, gender and biochemical markers of fluid overload.

Using tracer dilution techniques, Bonfils et al and Abdlebreht et al (Bonfils P K et al., Impact of diuretic treatment and sodium intake on plasma volume in patients with compensated systolic heart failure. Eur Heart J 2010; 12:995-1001 and Adlbrecht C, Kommata S, et al. Chronic heart failure leads to an expanded plasma volume and pseudoanaemia, but does not lead to a reduction in the body's red cell volume. Eur Heart J 2008; 29: 2343-50.) reported actual PV values of 37±6 mL/kg and 37±4 mL/kg, respectively, in line with the calculated value of 37±3 mL/kg of the present study. This contrasts to the higher PV levels measured in decompensated CHF patients (58±3 mL/kg) and healthy controls (43±3 mL/kg) (Anand I S, Ferrari R, et al. Edema of cardiac origin. Studies of body water and sodium, renal function, hemodynamic indexes, and plasma hormones in untreated congestive cardiac failure. Circulation 1989; 80: 299-305.) and reflects the impact of optimal medical and device-based therapies on volume status. Consistent with the aforementioned PV levels, and the fact that the ideal PV equation was originally derived in normal individuals, it is unsurprising that patients in the FAIR-HF study had mean PV levels that were ˜5% lower than their estimated ideal.

Thus, in a further aspect of the invention, the patients to be treated are characterized by having an increased relative PVS of at least −3.5%, preferably at least −4%, preferably at least −4.5%, preferably at least −5%.

In a further aspect of the invention the treatment results in a change of relative PVS by about at least 1.0% point, preferably by about 1.5% points, preferably by about 2.0% points, preferably by about 2.5% points, preferably by about 3.0% points, more preferably by about 3.5% points. Ideally the PVS is normalized to a calculated value of 0%±1.0%.

The relative plasma volume status (PVS) is used as a novel index for determining congestion, which indicates the degree to which subjects have deviated from their ideal PV. The novel PVS index is calculated with the equations shown below and accordingly, a further aspect of the invention relates to a method of determining the relative plasma volume status (PVS) as an index of the degree of congestion by applying the following calculation:

a) calculating the actual plasma volume (PV) of a subject with the formula:

actual PV=([1−Hct]×[a+(b×weight [kg])])
wherein
Hct is the measured hematocrit fraction,
a=1530 in males and 864 in females,
b=41 in males and 47.9 in females;

b) calculating the ideal PV with the formula:

ideal PV=c×weight (kg)
wherein
c=39 in males and 40 in females,
weight (kg)=the subject's body weight (kg);
and

c) calculating the relative PVS with the formula:

PVS=([actual PV−ideal PV]/ideal PV)×100%.

This contraction of PV in CHF is important, as it likely allows the failing myocardium to continue to operate on a reasonable portion of the Frank-Starling curve.

Hence, administration of an intravenous iron delivery product, namely of intravenous ferric carboxymaltose, has been found to have a positive effect on the function of the heart in patients suffering from heart failure and iron deficiency, more specifically patients suffering from chronic heart failure and iron deficiency, yet more specifically patients suffering from chronic heart failure with reduced left ventricular ejection fraction (LVEF) and iron deficiency, yet more specifically with chronic heart failure with LVEF 35% and iron deficiency.

This positive effect of an intravenous ferric carboxymaltose on the function of the heart is also deducible from the positive effect the product has been shown to have on clinical congestion. It is conceivable that FCM might directly modulate key aspects of the CHF syndrome such as the neurohormonal axis to trigger decongestion. This is plausible given that iron deficiency enhances circulating catecholamine levels which promote clinical congestion partly by activating the renin-angiotensin-aldosterone cascade. Irrespective of the underlying mechanism(s), changes in relative PVS over time correlate to alterations in other indices of CHF status in terms of heart function.

Further, it is known that congestion also has an effect on the heart's structural parameters leading to ventricular remodeling (G. Parrinello in Heart Fail. Rev., January 2015, 20(1), 13-24 and Mihai Gheorghiade, M D et al. in the “The American Journal of Medicine (2006)” Vol 119 (12A), S3-S10 —“Congestion in Acute Heart Failure Syndromes: An Essential Target of Evaluation and Treatment). Intracardiac pressures due to volume overload may begin to rise days to three weeks prior to the development of symptoms or weight gain. Some studies have suggested that in patients with pulmonary congestion, fluid overload is caused by fluid redistribution because of an increased vascular resistance/stiffness which may lead to both reduced capacitance in the large veins and increased arterial resistance with consequent endogenous fluid shift from the splanchnic bed into effective circulating volume rather than by endogenous fluid gain. Fluid redistribution and fluid accumulation may be variably combined in such patients. However, aside from this potential redistribution, true accumulation of fluid due to sodium and water retention secondary to adaptive neurohormonal changes is also at play. Congestion can increase LV (left ventricular) wall stress, functional mitral regurgitation and neurohormonal/inflammatory activation, thus exacerbating myocardial remodeling (chamber dilatation, increased ventricular sphericity and aggravated ischemia), loss of myocardial cells (reduced myocardial performance), decreasing ventricular function and leading to worsening hemodynamics and progressive HF.

Thus, in a second aspect of the invention, there is provided intravenously administered ferric carboxymaltose for use in the prevention or delay of structural changes of the heart associated with congestion in a patient suffering from heart failure with iron deficiency or in a patient being iron deficient and at risk of developing heart failure.

Preferably, the structural changes of the heart associated with congestion are selected from ventricular remodeling, myocardial remodeling or loss of myocardial cells.

In a third aspect of the invention, there is provided intravenously administered ferric carboxymaltose for use in the treatment of impaired function of the heart associated with congestion or deterioration of the function of the heart associated with congestion in a patient suffering from heart failure with iron deficiency or in a patient being iron deficient and at risk of developing heart failure.

Impaired or deteriorating heart function is defined as the heart's increasing inability to provide the absolute or relative blood volume that is required to meet the oxygen demand of the peripheral organs and is measured by determining the heart's ejection fraction. The ejection fraction is the fraction of blood pumped out of a ventricle of the heart with each heartbeat into the periphery of the body. It is typically measured by echocardiography and helps to determine the severity and the type of HF in a patient. All three types of ejection fraction, i.e. reduced EF (<40%), mid-range EF (40-49%) and preserved EF (50-100%) can occur in HF patients.

The treatment of the impaired or deteriorating function of the heart associated with congestion comprises the improvement of the impaired or deteriorating function of the heart, the delay of the onset of the impaired or deteriorating function of the heart, the delay of recurrence of the impaired or deteriorating function of the heart, the delay of deterioration of the impaired or deteriorating function of the heart, the prevention of onset of the impaired or deteriorating function of the heart, the prevention of recurrence of the impaired or deteriorating function of the heart and/or the prevention of deterioration of the impaired or deteriorating function of the heart.

Preferably, the impaired function of the heart is selected from left-ventricular impairment or dysfunction (left-ventricular heart failure, LVHF) or right-ventricular impairment or dysfunction (right-ventricular heart failure, RVHF) or biventricular impairment or dysfunction with both the left and the right ventricles being impaired or dysfunctional.

Therefore, the intravenous administration of ferric carboxymaltose is a very attractive new therapeutic option to treat a patient suffering from heart failure with iron deficiency or a patient being iron deficient and at risk of developing heart failure.

Thus, in a fourth aspect of the invention, there is provided intravenously administered ferric carboxymaltose for use as a diuretic in the treatment of heart failure in a patient suffering from heart failure with iron deficiency or in a patient being iron deficient and at risk of developing heart failure.

More specifically, there is provided intravenously administered ferric carboxymaltose for use as a diuretic, in particular in the treatment of heart failure in a patient suffering from heart failure with iron deficiency or in a patient being iron deficient and at risk of developing heart failure, wherein said treatment of heart failure comprises the treatment or prevention of congestion associated with the impaired or deteriorating function of the heart.

Yet more specifically, there is provided intravenously administered ferric carboxymaltose for use as a diuretic in the treatment of heart failure in a patient suffering from heart failure with iron deficiency or in a patient being iron deficient and at risk of developing heart failure, wherein said treatment of heart failure comprises the treatment or prevention of congestion associated with the impaired or deteriorating function of the heart, said impaired or deteriorating function of the heart being selected from left ventricular impairment or dysfunction, right ventricular impairment or dysfunction or biventricular impairment or dysfunction.

Further, there is provided intravenously administered ferric carboxymaltose for use as a diuretic in the treatment of heart failure in a patient suffering from heart failure with iron deficiency or in a patient being iron deficient and at risk of developing heart failure, wherein said treatment of heart failure comprises the treatment or prevention of congestion associated with the impaired or deteriorating function of the heart, said impaired or deteriorating function of the heart being selected from heart failure with a preserved ejection fraction (HFpEF), heart failure with a mid-range ejection fraction (HFmrEF) or heart failure with a reduced ejection fraction (HFrEF).

In a particularly preferred embodiment there is described intravenously administered ferric carboxymaltose for use as a diuretic in the treatment of heart failure in a patient suffering from heart failure with iron deficiency, whereby heart failure is heart failure with a reduced ejection fraction. More specifically, there is described intravenously administered ferric carboxymaltose for use as a diuretic in the treatment of heart failure in a patient suffering from heart failure with iron deficiency, whereby heart failure is chronic heart failure with a reduced ejection fraction, which is also termed chronic systolic heart failure.

In a particularly preferred embodiment there is described intravenously administered ferric carboxymaltose for use as a diuretic in the treatment of heart failure in a patient suffering from heart failure with iron deficiency and congestion, whereby heart failure is heart failure with a reduced ejection fraction. More specifically, there is described intravenously administered ferric carboxymaltose for use as a diuretic in the treatment of heart failure in a patient suffering from heart failure with iron deficiency and congestion, whereby heart failure is chronic heart failure with a reduced ejection fraction, which is also termed chronic systolic heart failure.

Further, there is provided intravenously administered ferric carboxymaltose for use as a diuretic in the treatment of heart failure in a patient suffering from heart failure with iron deficiency or in a patient being iron deficient and at risk of developing heart failure, wherein said treatment of heart failure comprises the treatment or prevention of congestion in patients with impaired or deteriorating function of the heart, said impaired or deteriorating function of the heart being the result of a structural change of the heart associated with congestion.

Further, said intravenously administered ferric carboxymaltose can be used to prevent or delay cardiac remodeling in a patient suffering from heart failure with iron deficiency or in a patient being iron deficient and at risk of developing heart failure.

Heart failure in the context of the invention is either chronic, stable, decompensated, acute, de novo, compensatory, refractory or congestive heart failure.

Chronic heart failure is defined as a condition wherein the characteristic symptoms and signs of HF have been present for a certain period of time. The term ‘stable’ is used only when the symptoms are well-controlled for at least one month. The worsening of chronic stable HF is termed ‘decompensated HF,’ while a sudden aggravation or failure leading to hospitalization is termed ‘acute HF.’ The initial development of HF symptoms due to illnesses such as myocardial infarction or myocarditis is referred to as ‘de novo HF.’ The term ‘compensatory HF’ is used in HF patients who are asymptomatic or show an improvement in their condition for a certain period of time. The term ‘congestive HF,’ used more often in the United States, indicates acute and chronic HF with signs of salt or fluid retention.

In particular, the current invention provides intravenously administered ferric carboxymaltose for use as a diuretic in the treatment of congestion or of congestion associated with impaired function of the heart or of congestion associated with deterioration of the function of the heart in a patient suffering from heart failure with iron deficiency or being iron deficient and at risk of developing heart failure, wherein heart failure is either chronic or acute heart failure.

Further, there is provided intravenously administered ferric carboxymaltose for use as a diuretic in the treatment of heart failure in a patient suffering from heart failure with iron deficiency or being iron deficient and at risk of developing heart failure, wherein heart failure is either chronic or acute heart failure.

Also, there is provided intravenously administered ferric carboxymaltose for use as a diuretic in the prevention or delay of structural changes to the heart associated with congestion in a patient suffering from heart failure with iron deficiency or being iron deficient and at risk of developing heart failure, wherein heart failure is either chronic or acute heart failure.

While the FAIR-HF sub-study mentioned above shows the effects intravenous ferric carboxymaltose has on treatment of congestion in a patient suffering from heart failure with iron deficiency or in a patient being iron deficient and at risk of developing heart failure, a further clinical trial termed AFFIRM-AHF is studying the effect of IV ferric carboxymaltose in patients suffering from acute heart failure with iron deficiency. Apart from a number of other selection criteria, study participants display edema 1+ on a 0-3+ scale, indicating indentation of skin with mild digital pressure that requires 10 or more seconds to resolve in any dependent area including extremities or the sacral region. Thus, in a further aspect of the invention, the patients to be treated are characterized by displaying edema 1+ on a 0-3+ scale.

In a further aspect of the invention the treatment results in a reduction of edema to a value <1 on a 0-3+ scale.

Further, study participants display acute HF with an ejection fraction of below 50% (HFrEF or HFmrEF), i.e. acute systolic HF. While the primary endpoints of this study are aimed at investigating repeated HF hospitalizations and cardiovascular (CV) death up to 52 weeks after randomization of participants, parameters needed for the assessment of an effect of the treatment on clinical congestion and thus on the function of the heart are also collected and analyzed.

Chronic, acute, stable, decompensated, de novo, compensatory, refractory or congestive heart failure in the context of the invention are further left-ventricular impairment or dysfunction (left-ventricular heart failure, LVHF) or left-ventricular impairment or dysfunction and right-ventricular impairment or dysfunction (right-ventricular heart failure, RVHF). HF can also be biventricular with both the left and the right ventricles being impaired or dysfunctional.

Further, heart failure, wherein heart failure is left-ventricular impairment or dysfunction, right-ventricular impairment or dysfunction or biventricular impairment or dysfunction, can be classified as systolic or diastolic. Systolic HF indicates a left ventricle (LV) unable to contract normally resulting in reduced pumping function and output. Systolic HF results in a HF with a reduced ejection fraction (HFrEF), whereby ejection fraction is the fraction of blood pumped out of a ventricle of the heart with each heartbeat into the periphery of the body. The ejection fraction is calculated by dividing the stroke volume by the end-diastolic volume, i.e. it is a volumetric measure of the pumping efficiency of the heart. Stroke volume (SV) is the volume of blood pumped from the left ventricle per beat. End-diastolic volume (EDV) is the volume of blood in the ventricle at end load or filling (diastole). Diastolic HF is HF whereby the LV is unable to relax fully in the resting period between two heart beats resulting in reduced filling of the LV. Diastolic HF results in HF with a preserved ejection fraction (HFrEF). HF can also result in a mid-range ejection fraction (HFmrEF). HFrEF is characterized by a left-ventricular ejection fraction (LVEF)<40% of an ejection fraction present in the absence of HF, while HFpEF has a LVEF 50% and HFmrEF has a LVEF=40-49%. An ejection fraction is typically considered to be normal if it is 55%.

Iron delivery products are pharmaceutically acceptable solutions for use in the treatment of iron deficiency with or without anemia of an iron deficient and/or anemic patient. Examples of known suitable iron delivery products comprise iron delivery products from the group of ferric carboxymaltose, iron sucrose, iron dextran, sodium ferric gluconate, ferumoxytol, iron isomaltoside, iron gluconate and β-ferric oxihydroxide hydroxyethyl amylopectin glucoheptonic acid (polyglucoferron).

The inventors of the present invention surprisingly found that among such iron delivery products ferric carboxymaltose turned out to effectively act on the relative PVS in patients suffering from heart failure with iron deficiency or in patients being iron deficient and at risk of developing heart failure, thus being suitable as a new diuretic medicament.

Ferric carboxymaltose is an innovative non-dextran intravenous iron (i.v.) replacement therapy commercialized under the trademarks Ferinject® or Injectafer®. To date, Ferinject® has gained marketing authorization in more than 70 countries worldwide for the treatment of iron deficiency where oral iron is ineffective or cannot be used. In many countries, intravenous iron replacement products are primarily used to treat dialysis patients. However, iron deficiency can exist independently of other underlying medical conditions as well as being a common complication of many other illnesses. So far, ferric carboxymaltose has not been registered for any other indication than the treatment of iron deficiency.

Ferric carboxymaltose was developed for rapid intravenous (IV or i.v.) administration in high doses for the treatment of iron deficiency and the rapid infusion of up to 1000 mg of elemental iron of ferric carboxymaltose over 15 min has been shown to be well tolerated. The ferric carboxymaltose complex has a nearly neutral pH (5.0-7.0) with a physiologic osmolarity and no dextran cross-reactivity. The iron-carbohydrate ferric carboxymaltose complex is more stable than ferric gluconate or iron sucrose, permitting slow and controlled delivery of high doses of iron into target tissues.

The term ferric carboxymaltose covers water soluble iron(III)-carbohydrate complexes such as described in the European Patent EP 2287204 (incorporated by reference). In particular water soluble iron(III)-carbohydrate complexes on the basis of the oxidation products of maltodextrins, wherein the iron (III) carbohydrate complexes have a weight average molecular weight of 80 kDa to 400 kDa (as determined in particular by gel permeation chromatography (GPC) as described for example by Geisser et al. in Arzneim. Forsch/Drug Res. 42(11), 12, 1439-1452 (1992), paragraph 2.2.5.). Preferred ferric carboxymaltose compounds have a weight average molecular weight (Mw) of between 100 and 230 kDa and more preferably of between 120 and 180 kDa. A ferric carboxymaltose compound can also have a weight average molecular weight (Mw) of about 150 kDa. “Ferric carboxymaltose” is an INN (International nonproprietary name) or USAN (United States Adopted Names) for polynuclear iron(III)-oxyhydroxide carboxypolymaltose (carboxymaltodextrin) compounds (CAS REGISTRY NUMBER 1461680-64-7; (VIT-45; Ferinject® or Injectafer®)).

Intravenous administration in the context of the present invention can refer to an intravenous drip infusion or a slow bolus injection. Bolus injection should occur over at least one minute per 100 mg elemental iron administered. Intravenous infusion should occur over at least six minutes when 500 mg of elemental iron are administered in the form of ferric carboxymaltose, and at least 5 minutes or at least 15 minutes when 1000 mg of elemental iron are administered in the form of ferric carboxymaltose. The ferric carboxymaltose is administered in doses and time intervals shown in clinical trials to be safe for said product.

Overall doses of the ferric carboxymaltose per patient will typically vary between 100 mg to 2000 mg, preferably between 200 to 1500 mg or between 200 mg and 1000 mg or preferably between 500 to 1500 mg or between 500 mg and 1000 mg of elemental iron depending on the body weight (kg) of the patient and their Hb levels (g/dL) measured. Individual doses administered on this occasion can be 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg and 1000 mg of elemental iron, wherein individual doses can be combined in such a way as to result in an overall dose of between 200 to 2000 mg, preferably between 500 to 1500 mg or between 500 mg and 1000 mg or between 750 to 1000 mg or between 750 to 1500 mg, or preferably between 200 to 1000 mg.

In yet another clinical study named HEART-FID a new method of treatment of congestion, of congestion associated with impaired heart function, of congestion associated with deterioration of heart function, of heart failure and/or a method of treatment preventing or delaying harmful structural changes to the heart in a patient suffering from heart failure with iron deficiency is investigated by administering to the patient ferric carboxymaltose. Further, a method of preventing or delaying the onset of structural changes to the heart by administering to a patient suffering from heart failure with iron deficiency is investigated by administering to the patient ferric carboxymaltose. In said study, the patient receives a single dose of ferric carboxymaltose of 15 mg of elemental iron/kg body weight up to a maximum dose of 750 mg elemental iron on Day 0 and Day seven and, optionally, again after six months in cases of iron deficiency recurrence. Administration is repeated every six months for as long as heart failure with iron deficiency or a risk of developing heart failure with iron deficiency persists.

The method of treatment according to the study is further characterized in that administration of the ferric carboxymaltose occurs on the first day of treatment and again after seven days and after six months in cases of iron deficiency recurrence. Optionally, administration of the ferric carboxymaltose may additionally occur after four weeks, six weeks, 12 weeks, 16 weeks and/or 24 weeks. In yet another embodiment of the invention, the method of treatment is additionally characterized in that administration of the ferric carboxymaltose is repeated every six months for as long as heart failure with iron deficiency or a risk of developing heart failure with iron deficiency persists, preferably if heart failure is heart failure with reduced ejection fraction (HFrEF), more preferably HFrEF wherein EF 35% or EF 25%. Yet more preferably, HF is chronic HF with HFrEF, i.e. chronic systolic HF, wherein EF 35% or EF 25%.

There is thus provided in a fifth aspect of the invention a method of treatment of congestion in a patient suffering from heart failure with iron deficiency or being iron deficient and at risk of developing heart failure, wherein ferric carboxymaltose is intravenously administered to the patient.

Said treatment of congestion comprises the improvement of congestion, the maintenance of congestion, the delay of deterioration of congestion and/or the prevention of deterioration of congestion.

In a sixth aspect of the invention there is described a method of treatment of impaired function of the heart associated with congestion or a method of treatment of the deterioration of the function of the heart associated with congestion in a patient suffering from heart failure with iron deficiency or being iron deficient and at risk of developing heart failure, wherein ferric carboxymaltose is intravenously administered to the patient.

Said treatment of impaired heart function associated with congestion and the treatment of the deterioration of heart function comprises the improvement of heart function, the maintenance of heart function, the delay of the loss of heart function, the delay of the deterioration of heart function, the prevention of loss of heart function and the prevention of deterioration of heart function.

In a seventh aspect of the invention there is described a method of treatment of congestion associated with heart failure in a patient suffering from heart failure with iron deficiency or being iron deficient and at risk of developing heart failure, wherein ferric carboxymaltose is intravenously administered to the patient.

In an eighth aspect of the invention, there is provided a method of treatment, preventing or delaying harmful structural changes to the heart associated with congestion in a patient suffering from heart failure with iron deficiency or being iron deficient and at risk of developing heart failure, wherein ferric carboxymaltose is intravenously administered to the patient.

The above methods of treatment are characterized in that the impaired function of the heart associated with congestion, the deterioration of the function of the heart associated with congestion and the heart failure are selected from left-ventricular impairment or dysfunction (left-ventricular heart failure, LVHF), right-ventricular impairment or dysfunction (right. ventricular heart failure, RVHF) or biventricular impairment or dysfunction.

Further, heart failure in the above methods of treatment can be chronic, stable, decompensated, acute, de novo, compensatory, refractory or congestive. Preferably, it is acute or stable.

Heart failure in the above methods of treatment can further be systolic or diastolic and can be further characterized by a preserved ejection fraction (HFpEF), a mid-range ejection fraction (HFmrEF) or a reduced ejection fraction (HFrEF).

Any of the above methods of treatment or uses are used in a patient suffering from heart failure with iron deficiency and congestion.

Said intravenously administered ferric carboxymaltose in the treatment of congestion, of congestion associated with impaired function of the heart, of congestion associated with the deterioration of the function of the heart, of heart failure and in the treatment preventing or delaying harmful structural changes to the heart associated with congestion is a pharmaceutically acceptable solution of an iron compound suitable for intravenous administration, whereby said ferric carboxymaltose is suitable for use in the treatment of iron deficiency with or without anemia of an iron deficient and/or anemic patient.

Intravenous administration in the context of the present method can refer to an intravenous drip infusion or a slow bolus injection. Bolus injection should occur over at least one minute per 100 mg iron administered. The ferric carboxymaltose is administered in doses and time intervals shown in clinical trials to be safe for said product. Intravenous infusion should occur over at least six minutes when 500 mg of elemental iron are administered in the form of ferric carboxymaltose, and at least 5 minutes or at least 15 minutes when 1000 mg of elemental iron are administered in the form of ferric carboxymaltose.

Further, said methods of treatment are characterized in that the ferric carboxymaltose is administered in doses per patient of between 100 mg to 2000 mg, preferably between 100 mg to 1000 mg, between 200 to 1500 mg, between 200 to 1000 mg, between 500 to 1500 mg, between 500 mg and 1000 mg, between 750 to 1000 mg or between 750 to 1500 mg of elemental iron depending on the body weight (kg) of the patient and their Hb levels (g/dL) measured. Individual doses administered on this occasion can be 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg and 1000 mg of elemental iron, wherein individual doses can be combined in such a way as to result in an overall dose of between 200 to 2000 mg, preferably between 500 to 1500 mg or between 500 mg and 1000 mg or between 750 to 1000 mg or between 750 to 1500 mg, or between 200 to 1000 mg.

The above methods of treatment are further characterized in that administration of the ferric carboxymaltose occurs on the first day of treatment and after seven days, then, optionally, after four weeks, six weeks, eight weeks, 12 weeks, 16 weeks, 24 weeks, 36 weeks and/or six months. In yet another embodiment of the invention, the method of treatment is additionally characterized in that administration is repeated every six months for as long as heart failure with iron deficiency or a risk of developing heart failure with iron deficiency persists. Preferably, heart failure in this method of treatment is heart failure with preserved ejection fraction (HFpEF), with mid-range ejection fraction (HFmrEF) or with reduced ejection fraction (HFrEF). More preferably, heart failure in this method is HFrEF, specifically wherein EF is 50%, yet more specifically wherein EF 35% or EF 25%.

The present invention further comprises the following embodiments:
1. Intravenously administered ferric carboxymaltose for the use in the treatment of congestion or impaired function of the heart associated with congestion or deterioration of the function of the heart associated with congestion in a patient suffering from heart failure with iron deficiency or in a patient being iron deficient and at risk of developing heart failure.
2. Intravenously administered ferric carboxymaltose for the use according to 1, wherein the impaired function of the heart associated with congestion or the deterioration of the function of the heart associated with congestion is selected from left-ventricular impairment or dysfunction (left-ventricular heart failure, LVHF) of the heart or right-ventricular impairment or dysfunction (right-ventricular heart failure, RVHF) of the heart or biventricular impairment or dysfunction of the heart.
3. Intravenously administered ferric carboxymaltose for the use according to 1 and 2, wherein the treatment comprises the improvement, the delay of onset, the delay of recurrence, the delay of deterioration, the prevention of onset, the prevention of recurrence and/or the prevention of deterioration of congestion, of the impaired function of the heart and/or of the deterioration of the heart.
4. Intravenously administered ferric carboxymaltose for the use according to 1 to 3, wherein heart failure is either chronic or acute heart failure.
5. Intravenously administered ferric carboxymaltose for the use according to 4, wherein heart failure is either systolic or diastolic.
6. Intravenously administered ferric carboxymaltose for the use according to 4 or 5, wherein heart failure is characterized by a preserved ejection fraction (HFpEF), a mid-range ejection fraction (HFmrEF) or a reduced ejection fraction (HFrEF).
7. Intravenously administered ferric carboxymaltose for the use according to any of the preceding 1 to 6, wherein the iron product is administered with a dose of between 100 mg to 2000 mg of elemental iron, preferably of between 200 mg and 1500 mg or between 200 mg and 1000 mg of elemental iron, or preferably of between 500 mg and 1500 mg or between 500 mg and 1000 mg of elemental iron or a dose of 100 mg, 200 mg, 500 mg, 750 mg or 1000 mg of elemental iron.
8. Intravenously administered ferric carboxymaltose for the use according to any of the preceding 1 to 7, wherein administration occurs on the first day of treatment and after seven days, and, optionally, after four weeks, six weeks, 12 weeks, 16 weeks, 24 weeks and/or six months.
9. Intravenously administered ferric carboxymaltose for the use according to 8, wherein administration is repeated every six months for as long as heart failure with iron deficiency or a risk of developing heart failure with iron deficiency persists.
10. A method of treatment of congestion, of the deterioration of heart function associated with congestion and/or of the loss of heart function associated with congestion in a patient suffering from heart failure with iron deficiency or being iron deficient and at risk of developing heart failure, wherein an intravenously administered ferric carboxymaltose is administered to the patient.
11. A method of treatment according to 10, wherein the impaired function of the heart associated with congestion or the deterioration of the function of the heart associated with congestion is selected from left-ventricular impairment or dysfunction (left-ventricular heart failure, LVHF) of the heart or right-ventricular impairment or dysfunction (right-ventricular heart failure, RVHF) of the heart or biventricular impairment or dysfunction of the heart.
12. A method of treatment according to 10 or 11, wherein a dose of between 100 mg to 2000 mg of elemental iron, preferably of between 200 mg and 1500 mg or between 200 mg and 1000 mg of elemental iron, or preferably of between 500 mg and 1500 mg or between 500 mg and 1000 mg of elemental iron or a dose of 100 mg, 200 mg, 500 mg, 750 mg or 1000 mg of elemental iron is administered to the patient in the form of ferric carboxymaltose.
13. A method of treatment according to 12, wherein administration occurs on the first day of treatment and after seven days, and, optionally, after four weeks, six weeks, 12 weeks, 16 weeks, 24 weeks and/or six months.
14. A method of treatment according to 13, wherein administration is repeated every six months for as long as heart failure with iron deficiency or a risk of developing heart failure with iron deficiency persists.

### Examples

### Study Designs

### FAIR-HF Plasma Volume Status (PVS) Substudy

The design and primary results of the FAIR-HF trial (Study ID Numbers: FER-CARS-02; ClinicalTrials.gov Identifier: NCT00520780) have been reported previously (Anker S D, Comin Colet J, et al. Ferric carboxymaltose in patients with heart failure and iron deficiency. N Engl J Med. 2009; 361:1-13 and Anker S D, Colet J C et al.; FAIR-HF committees and investigators. Rationale and design of Ferinject assessment in patients with IRon deficiency and chronic Heart Failure (FAIR-HF) study: a randomized, placebo-controlled study of intravenous iron supplementation in patients with and without anaemia. Eur J Heart Fail. 2009 November; 11(11):1084-91.)

FAIR-HF is a multi-centre, randomized, double-blind trial including 459 ambulatory patients with NYHA class II or III CHF, a left ventricular ejection fraction (LVEF) ≤40% (NYHA II) or ≤45% (NYHA III), iron deficiency (ID) as defined by a ferritin <100 ug/l or 100 to 299 ug/l with a transferrin saturation (TSAT)<20%, and a hemoglobin (Hb) level between 9.5 and 13.5 g/dl. Patients were randomized in a 2:1 ratio to intravenous (IV) iron as ferric carboxymaltose (FCM; Ferinject®) or IV placebo (saline) weekly until iron repletion (correction phase), then 4 weekly until week 24 (maintenance phase). FCM was administered in doses of 200 mg (4 mL) weekly up to iron repletion (correction phase of variable duration depending on individual iron deficit). The calculated dose was rounded to the next 100 mg iron, i.e. the final dose may be 100 mg iron depending on the individual iron deficit. After the correction phase, FCM was administered monthly in doses of 200 mg until the 24th week (maintenance phase).

In the placebo group, during the correction phase, patients will receive the number of normal saline injections (4 mL weekly) corresponding to the calculated total iron dose needed according to the individual iron deficit. During the maintenance phase, placebo patients will receive 4 mL normal saline monthly. Regular assessments were done at weeks 4, 12, and 24. After 24 weeks of treatment, patients randomized to IV FCM had significant improvements in Patient Global Assessment (PGA), NYHA class, and 6 minute walking distance (6MWD), compared to patients allocated to saline.

The hematocrit value (Hct or Ht) and weight at randomization were used for plasma volume status (PVS) calculations and were available in 436 (95%) patients whose data were therefore used in this post-hoc analysis.

### Plasma Volume Equations

Actual plasma volume (PV) was calculated with the following equation that was derived by curve-fitting techniques using subjects' Hct and weight compared with PV values measured with gold standard radioisotope assays:

actual PV=([1−Hct]×[a+(b×weight [kg])])

where Hct is a fraction and a=1530 in males and 864 in females, and b=41 in males and 47.9 in females.

Ideal PV was calculated from the following well established formula:

ideal PV=c×weight (kg)

where c=39 in males and 40 in females

Relative PVS, an index of the degree to which patients have deviated from their ideal PV, was subsequently calculated from the following equation:

PVS=([actual PV−ideal PV]/ideal PV)×100%

### Clinical Congestion

Patients were weighed and examined for signs and symptoms of congestion at randomization and at weeks 4, 12 and 24. The presence or absence of peripheral edema, pulmonary congestion (crackles, rales), and an S3 gallop (third heart sound) were recorded, as was the actual jugular venous pressure (JVP).

### Statistics

Data are presented as mean±SD, frequencies (%), or medians (interquartile range [IQR]). Intergroup comparisons were made using a Kruskall-Wallis analysis of variance (ANOVA), analysis of covariance (ANCOVA), Student t-test, Pearson χ2 test, or a Mann Whitney U-test as appropriate. Associations between variables were determined using logistic regression. A P-value <0.10 was used to enter and retain covariates into stepwise multivariable regression models.

To assess the impact of treatment assignment on PVS, comparisons of changes in PVS from baseline at 4, 12, and 24 weeks between the placebo and FCM group were evaluated by comparing the least square means at each visit from a repeated-measures model using an unstructured covariance structure and adjusting for the continuous baseline value and a visit x drug interaction. Changes over time for other variables were similarly determined.

For prognostic analyses, the key safety endpoint was the combined outcome of all cause death or hospitalization. The relation between safety endpoints and baseline PVS as a continuous or categorical (≤−4% vs. >−4%) variable were determined using Cox proportional hazards analyses. This categorical cut-off was previously shown to best stratify outcomes in a large CHF cohort.10 In Cox analyses, the proportional hazard assumption was assessed by inspection of the log time-log hazard plot. The event rate using a person-time ‘at risk’ denominator, hazard ratio (HR), 95% confidence interval (CI), and significance levels for χ2 (likelihood ratio test) were calculated. Kaplan-Meier cumulative survival plots were constructed for display and assessed using the log-rank test.

Data were analyzed using SAS version 9.2 (SAS Institute Inc, Cary, N.C.). A 2-tailed P-value <0.05 was considered statistically significant.

### Results

### Calculated PVS in FAIR-HF and its Baseline Correlates

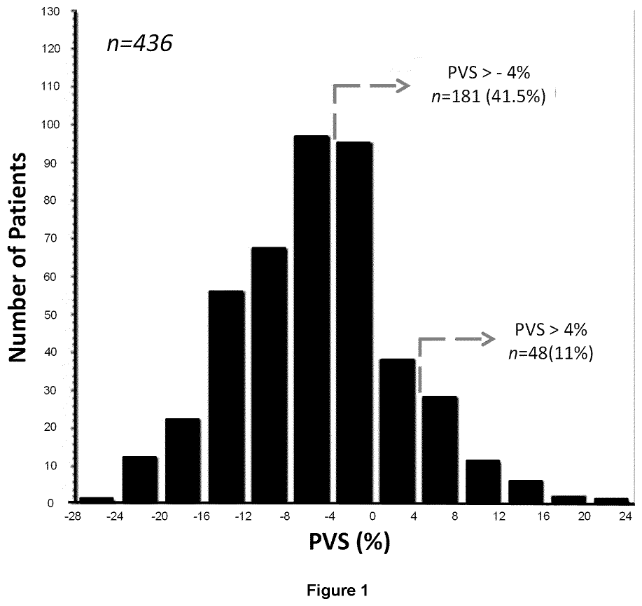

Baseline characteristics of the FAIR-HF cohort have previously been published. The clinical and laboratory features of patients at enrolment were similar between those randomized to FCM (n=304) and those randomized to placebo (n=155). The baseline characteristics of the 436 patients included in this sub-study are shown in Table 2. Their mean weight-adjusted actual plasma volume (PV), weight-adjusted ideal PV, and PVS were 37.3±3 mL/kg, 3036.6±573.2 mL/kg, and −5.5±7.7%, respectively. Values of plasma volume status (PVS) ranged from −25% to +24%. A PVS ≤0 or >0 was evident in 350 (80.3%) and 86 (19.7%) patients, respectively (FIG. 1). The mean jugular venous pressure (JVP) was 1.9 cm with 9.2% of patients having an elevated JVP (>4 cm), and 35%, 23%, and 12% of patients having peripheral edema, pulmonary edema, and an S3 gallop, respectively.

On stratification, patients with a PVS >0% tended to be older and female with biochemical markers of potentially greater fluid retention such as a lower serum albumin, higher blood urea nitrogen, and a lower estimated glomerular filtration rate (eGFR) than those with a PVS ≤0% (Table 2). They also had poorer haemodynamics and less premorbid hypertension. In a multivariable model incorporating age, gender, systolic blood pressure, albumin, eGFR, LVEF, and the use of diuretics, angiotensin converting enzyme (ACE) inhibitors and beta-blockers, only age (odds ratio 0.97, P=0.02), systolic blood pressure (odds ratio 1.02, P=0.01) and serum albumin (odds ratio 1.13, P=0.01) independently predicted PVS.

In Table 2, Data are reported as means (SD) or numbers (frequency) of patients. NYHA indicates New York Heart Association; CABG, Coronary artery bypass grafting; PTCA, Percutaneous coronary angioplasty; eGFR, estimated glomerular filtration rate; MDRD, modification of diet in renal disease equation; CRP, C-reactive protein; ACE, angiotensin converting enzyme inhibitor.

TABLE 2

| | All | | PVS ≤0% | | PVS >0% | | |
|---|---|---|---|---|---|---|---|
| | (N = 436) | | (N = 350) | | (N = 86) | | P Value |
| Clinical variables | | | | | | | |
| Age, years | 67.8 | (10.71) | 67.1 | (10.54) | 70.5 | (11) | 0.008 |
| Male gender, n (%) | 203 | (46.6) | 154 | (44.0) | 49 | (57.0) | 0.040 |
| NYHA III, n (%) | 361 | (82.8) | 288 | (82.3) | 73 | (84.9) | 0.635 |
| Left ventricular ejection fraction, % | 32.3 | (5.7) | 32.5 | (5.61) | 31.2 | (5.94) | 0.055 |
| Ischaemic aetiology, n (%) | 351 | (80.5) | 283 | (80.9) | 68 | (79.1) | 0.761 |
| Prior CABG, n (%) | 46 | (10.6) | 34 | (9.7) | 12 | (14.0) | 0.326 |
| Prior PTCA, n (%) | 61 | (14.0) | 48 | (13.7) | 13 | (15.1) | 0.862 |
| Prior stroke, n (%) | 29 | (6.7) | 24 | (6.9) | 5 | (5.8) | 0.815 |
| History of atrail fibrillation, n (%) | 131 | (30.0) | 107 | (30.6) | 24 | (27.9) | 0.695 |
| History of hypertension, n (%) | 352 | (80.7) | 291 | (83.1) | 61 | (70.9) | 0.014 |
| History of diabetes mellitus, n (%) | 120 | (27.5) | 103 | (29.4) | 17 | (19.8) | 0.080 |
| Physical examination | | | | | | | |
| Systolic blood pressure, mm Hg | 126.1 | (14.71) | 76.4 | (9.43) | 713 | (11-55) | 0.008 |
| Diastolic Mood pressure, mm Hg | 76.4 | (9.43) | 77.1 | (8.92) | 73.5 | (10.85) | 0.001 |
| Heart rate, bpm | 71.3 | (11.58) | 71.5 | (11.74) | 70.2 | (10.92) | |
| Weight, kg | 76.9 | (14.73) | 79.8 | (14.9) | 65 | (10.36) | <0.001 |
| Body mass index, kg/m² | 27.9 | (4.78) | 28.8 | (4.62) | 24.3 | (3.68) | <0.001 |
| Hip/waist ratio, cm | 1.1 | (0.15) | 1.1 | (0.15) | 1.1 | (0.15) | 0.289 |
| Jugular venous pressure, cm | 1.9 | (2.22) | 1.9 | (2-115) | 1.8 | (2.49) | 0.871 |
| Peripheral edema, n (%) | 152 | (34.9) | 127 | (36.3) | 25 | (29 1) | 0.255 |
| Pulmonary edema, n (%) | 99 | (22.7) | 80 | (22.9) | 19 | (22.1) | 0.883 |
| Third heart sound (S3 gallop), n (%) | 53 | (12.2) | 44 | (12.6) | 9 | (10.5) | 0.714 |
| Six-minute walk test distance, meters | 270.8 | (106.83) | 275 | (106.4) | 253.6 | (107.43) | 0.095 |
| Quality of Life | | | | | | | |
| KCCQ overall score | 59 | (20.05) | 58.4 | (20.16) | 61.7 | (19.48) | 0.173 |
| EQ-5D health score | 54.2 | (16.27) | 54.2 | (16.25) | 54 | (16.49) | 0.922 |
| Laboratory measurements | | | | | | | |
| Hemoglobin, g/dL | 11.9 | (1.29) | 12.2 | (1.18) | 10.9 | (1.14) | <0.001 |
| Hematocrit | 0.4 | (0.04) | 0.4 | (0.04) | 0.3 | (0 .03) | <0.001 |
| Serum ferritin, ug/L | 53.8 | (57.8) | 54.5 | (59.31) | 51.1 | (51.39) | 0.064 |
| Transferrin saturation, % | 17.4 | (11.25) | 17.7 | (9.92) | 16.1 | (15.55) | 0.242 |
| C-reactive protein, mg/L | 5 | (4.33) | 4.9 | (4.33) | 5.2 | (4.34) | 0.577 |
| Sodium mmg/L | 140.7 | (2.83) | 140.8 | (2.87) | 140.4 | (2.63) | 0.221 |
| Serum albumin, g/L | 43.1 | (2.78) | 43.3 | (2.72) | 42.2 | (2.83) | 0.001 |
| Alanine aminotransferase, U/L | 19.8 | (11.14) | 20.2 | (11.31) | 18.5 | (10.42) | 0.231 |
| [symbol] U/L | 22.8 | (9.58) | 22.7 | (9.67) | 23.1 | (9.29) | 0.752 |
| Creatinine mg/dL | 1.2 | (0.59) | 1.1 | (0.43) | 1.5 | (0.96) | <0.001 |
| eGFR (MDRD), mL/min/1.73 m² | 63.7 | (22.57) | 65.1 | (21.47) | 58.1 | (25.98) | 0.010 |
| Blood urea nitrogen, mg/dL | 24.5 | (12.87) | 23.4 | (11.81) | 28.8 | (15.86) | 0.001 |
| Calculated plasma volumes | | | | | | | |
| Ideal plasma volume, mL | 3037 | (573) | 3153 | (550) | 2553 | (395) | <0.001 |
| Actual plasma volume, mL | 2843 | (441) | 2373 | (445) | 2693 | (394) | 0.001 |
| Plasma volume status (PVS), % | −5.5 | (7.71) | −8.2 | (5.6) | 5.5 | (4.71) | — |
| Medications, % | | | | | | | |
| ACE inhibitor, n (%) | 402 | (92.2) | 326 | (93.1) | 76 | (88.4) | 0.1751 |
| β-blocker, n (%) | 373 | (85.6) | 299 | (85.4) | 74 | (86.0) | 1.000 |
| Aldosterone atagonist, n (%) | 211 | (43.4) | 167 | (47.7) | 44 | (51.2) | 0.6303 |
| Cardiac glycosides, n (%) | 69 | (15.8) | 52 | (14.9) | 17 | (19.8) | 0.3217 |
| Diuretic, n (%) | 402 | (92.2) | 323 | (92.3) | 79 | (91.9) | 1.000 |

[symbol]

indicates data missing or illegible when filed

### Impact of Ferric Carboxymaltose on Calculated PVS and Clinical Congestion

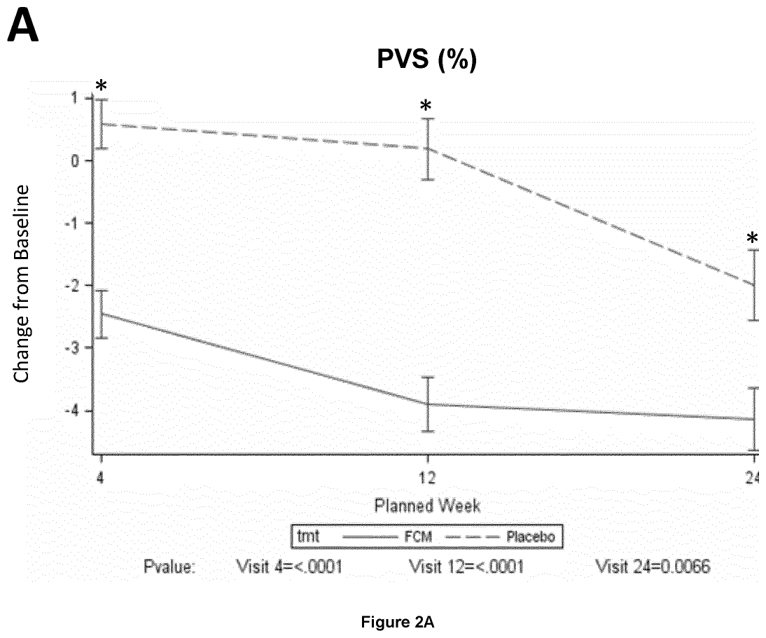

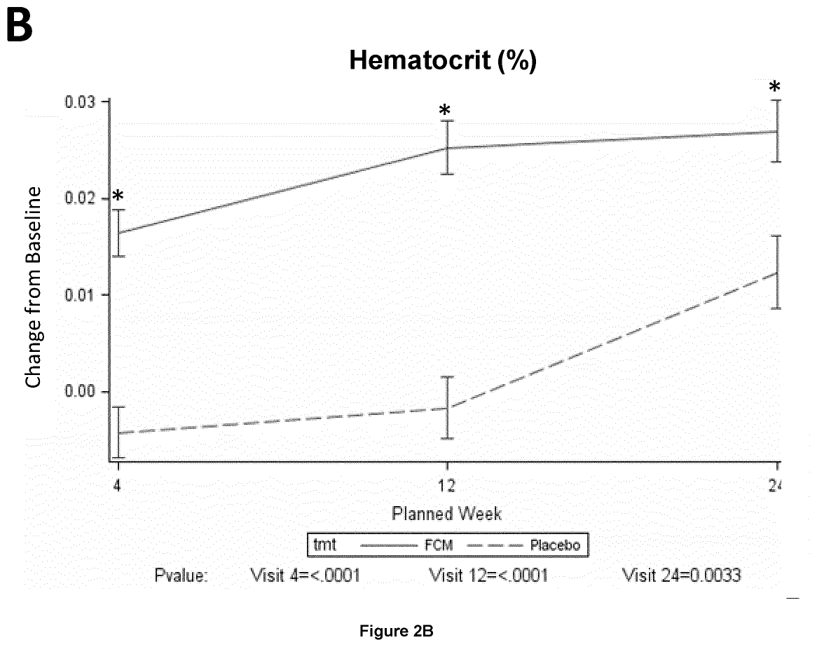

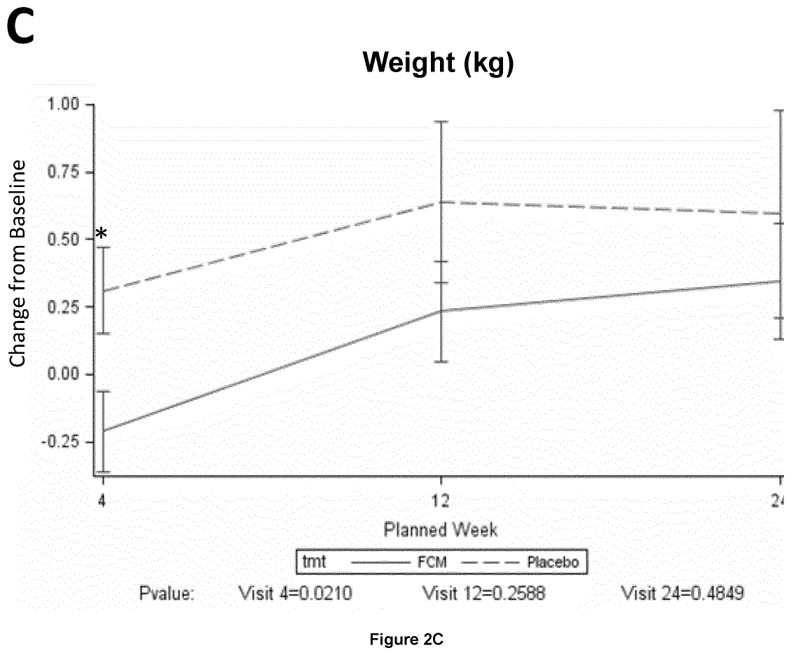

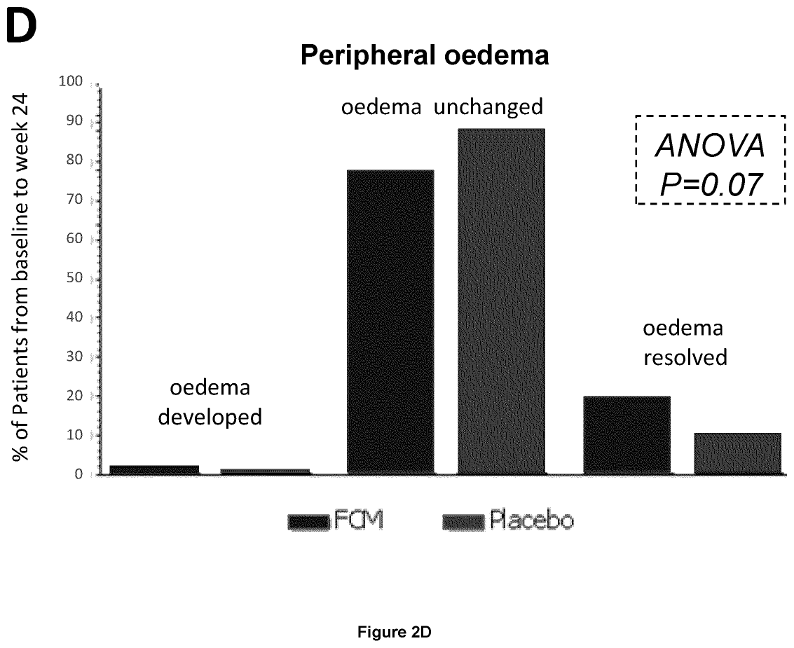

At baseline, mean PVS was similar in the FCM (−5.3%±7.7%) and placebo (−6%±7.8%) groups. Over time, treatment with FCM was associated with significant reductions from baseline in PVS at week 4 (−2.5±5.9% vs. 0.6±4.3%), 12 (−3.9±6.8% vs. 0.2±5.3%), and 24 (−4.1±7.4% vs. −2.0±5.5%), compared to placebo (FIG. 2a). The treatment effect was maximal at week 12. Treatment with FCM was also associated with significant increases from baseline in Hct at week 4 (0.02±0.04% vs. −0.004±0.03%), 12 (0.03±0.04% vs. −0.002±0.04%), and 24 (0.03±0.05% vs. 0.01±0.04%; FIG. 2b). In contrast, FCM treatment was associated with a significant reduction from baseline in weight at week 4 (−0.2±2.4 kg vs. 0.3±1.8 kg), but not at week 12 (0.2±3.0 kg vs. 0.6±3.3 kg), or 24 (0.3±3.2 kg vs. 0.6±3.7 kg; FIG. 2c). Similar to the effect on PVS, there was an improvement in peripheral edema with FCM treatment compared to placebo (FIG. 2d). Similar improvements in jugular venous distension (P=0.54), pulmonary edema (P=0.35) or the presence of an S3 gallop rhythm (P=1.00) with FCM treatment were not evident.

### Clinical Correlates of Change in Calculated PVS

A total of 309 (70.9%) patients survived to week 24 and had sufficient data to calculate change in PVS. From baseline to week 24, mean change in PVS (ΔPVS) in this cohort was −3.5±6.9%, with volume expansion (ΔPVS >0%) evident in 93 (30.1%) patients. Compared to those with volume expansion, patients with decreasing volume status had greater improvements in exercise capacity as reflected by 46MWD (61.4 meters vs. 43.5 meters, P=0.02) and were more likely to improve their NYHA status by at least 1 class (33.3% vs. 15.1%, P=0.001). On stratified or adjusted analyses, these differences were evident in both treatment groups.

### Calculated PVS and Outcomes in FAIR-HF

Safety endpoints are listed in Table 3. During the study period, 55 (13%) patients experienced the key safety endpoint of all-cause death or hospitalization, 9 (2%) died from any cause, 8 (2%) experienced a cardiovascular death, 3 (1%) experienced a CHF death, 46 (11%) were hospitalized for any cause, and 25 (6%) died or were hospitalized for worsening CHF.

TABLE 3

| | PVS ≤ −4% | | PVS > −4% | | | |
|---|---|---|---|---|---|---|
| | (n = 255) | | (n = 181) | | | |
| | Number | Incidence/ | Number | Incidence/ | | |
| | of | (100 patient- | of | (100 patient- | Hazard Ratio | |
| Endpoint of Event | Events | yr at risk) | Events | yr at risk) | 95% CI | P value |
| Any hospitalisation or death | 23 | 20 (16.7) | 32 | 25 (31.6) | 1.89 (1.05-3.40) | 0.031 |
| Any death | 3 | 3 (2.4) | 6 | 6 (7.1) | 2.94 (0.74-11.76) | 0.11 |
| Cardiovascular death | 3 | 3 (2.4) | 5 | 5 (5.9) | 2.45 (0.58-10.25) | 0.21 |
| Death due to worsening CHF | 1 | 1 (0.8) | 2 | 2 (2.4) | 2.93 (0.27-32.35) | 0.36 |
| Any hospitalisation | 20 | 17 (14.2) | 26 | 21 (26.5) | 1.86 (0.98-3.53) | 0.053 |
| Any hospitalisation for any | 15 | 14 (11.6) | 18 | 14 (17.3) | 1.48 (0.71-3.11) | 0.30 |
| cardiovascular reason | | | | | | |
| Any hospitalisation for | 7 | 6 (4.9) | 9 | 7 (8.4) | 1.74 (0.58-5.18) | 0.31 |
| worsening CHF | | | | | | |
| Any hospitalisation for any | 18 | 17 (14.1) | 24 | 18 (22.2) | 1.57 (0.81-3.05) | 0.18 |
| cardiovascular reason or death | | | | | | |
| Any hositalisation for worsening | 10 | 9 (7.3) | 15 | 12 (14.4) | 1.98 (0.84-4.71) | 0.11 |
| CHF or death | | | | | | |
| Any hospitalisation or death for | 18 | 17 (14.1) | 23 | 17 (21.0) | 1.48 (0.76-2.91) | 0.25 |
| any cardiovascular reason | | | | | | |
| Any hospitalisation or death | 8 | 7 (5.7) | 11 | 8 (9.6) | 1.70 (0.62-4.69) | 0.30 |
| for worsening CHF | | | | | | |

Patients with a baseline PVS >−4% experienced a higher incidence of all safety endpoints than those with a PVS ≤−4%, with the difference in incidence statistically significant for the key safety endpoint of all-cause death or hospitalization. In Cox analyses, a PVS >−4% was associated with an 89% (unadjusted HR 1.89, 95% CI 1.05 to 3.40, P=0.03) greater risk of the key endpoint (FIG. 3) even after bivariate adjustment for treatment assignment (adjusted HR 1.88, 95% CI 1.01 to 3.51, P=0.046), JVP (adjusted HR 2.18, 95% CI 1.05 to 4.51, P=0.04), or the presence of pulmonary (adjusted HR 1.88, 95% CI 1.01 to 3.50, P=0.047), or peripheral edema (adjusted HR 1.89, 95% CI 1.01 to 3.52, P=0.045). As a continuous variable, higher PVS values were also associated with a greater incidence of all-cause death or hospitalization (unadjusted HR 1.07, 95% CI 1.03 to 1.12, P<0.001).

### AFFIRM-AHF Study

AFFIRM-AHF is a multi-centre, randomized, double-blind, parallel-group placebo controlled trial with a fixed follow-up of 52 weeks per subject after randomization, whereby subjects are hospitalized for acute heart failure (AHF) and stabilized before randomization and are iron deficient, comparing the effect of intravenous (IV) ferric carboxymaltose (FCM) on hospitalizations and mortality in iron deficient patients admitted for acute heart failure. Approximately, 1100 randomized patients participate in this study.

### Treatment

Eligible subjects are randomized (1:1) to either IV FCM or placebo, i.e. a control treatment arm using IV NaCl 0.9%, using a validated centralized procedure (Interactive Web-based Randomization System (IWRS)). FCM is supplied in 10 mL vials with one 10 mL vial containing 500 mg iron as a sterile 5% weight/volume (w/v) iron solution in water for injection also containing the excipients sodium hydroxide, hydrochloric acid, water for injections. Study treatment is administered slowly as an undiluted bolus injection over at least 5 minutes for a 500 mg dose and over at least 15 minutes for a 1000 mg dose. The study treatment dose (mL) to be administered is determined by the subject's body weight and haemoglobin (Hb) value based on the dosing scheme per Table 4 describing the study treatment dosing regimen.

TABLE 4

| Treatment | Total FC M or Saline | | | | |
|---|---|---|---|---|---|
| Visit | Weight <70 kg | | Weight ≥70 kg | | Any Weight |
| | 8 g/dL⁽¹⁾ | 10 g/dL | 8 g/dL⁽¹⁾ | 10 g/dL | 14 g/dL |
| | ≤Hb | ≤Hb | ≤Hb | ≤Hb | <Hb |
| | <10 g/dL | ≤14 g/dL | <10 g/dL | ≤14 g/dL | ≤15 g/dL |
| Visit 2 | 2 × 10 mL | | | | 1 × 10 mL |
| (Week 0) | | | | | |
| Visit 3⁽²⁾ | 1 × 10 mL | No dose | 2 × 10 mL | 1 × 10 mL | No dose |
| (Week 6) | | | | | |
| Visit 4, | 1 × 10 mL | | | | |
| Visit 5 | (only if ID persists) | | | | |
| (Week 12, | | | | | |
| Week 24) | | | | | |

⁽¹⁾Following section in italics is applicable for The Netherlands only (NL only): The lower threshold of Hb values for subject eligibility prior to enrolment into the attain is set to 10 g/dL. Subjects enrolled in the study in NL only with Hb levels that are falling below the threshold of 10 g/dL during the study will need to be withdrawn from further study treatment dosing.
⁽²⁾Dosing at week 6 (Visit 3) will be based on the iron need upon screening Hb and weight values and only be done as subjects for whom Hb ≤15 g/dL.
Notes:
FCM = Fertic carboxymaltose;
Hb = Haemoglobin;
ID = Iron deficiency.

The study treatment doses administered at Visits 2 and 3 are considered as the “repletion phase” and any subsequent administrations (if ID persists) are considered as the “maintenance phase”. The first dose of study treatment is administered for all randomized subjects on the same day as randomization i.e., at Visit 2 while the subject is still hospitalized for the Index hospitalization after the acute care treatment of the index event and has been stabilized. The subsequent administrations of study treatment is done as part of the outpatient clinic visits at Week 6 (Visit 3), and at Weeks 12 (Visit 4) and 24 (Visit 5). Dosing at Week 6 (Visit 3) is based on the iron need upon screening Hb and weight values to replete iron as described in Table 4 and is only done in subjects for whom Hb ≤15 g/dL. Maintenance dosing at Weeks 12 (Visit 4) and 24 (Visit 5) is only for subjects in whom ID persists and for whom Hb ≥8 g/dL* and ≤15 g/dL at those visits. ID is defined as serum ferritin <100 ng/mL, or 100 ng/mL ≤serum ferritin ≤299 ng/mL if transferrin saturation (TSAT)<20%. The serum pregnancy test must also be negative for the respective visit for females of childbearing potential. Subjects are requested to return to the outpatient clinic for the administration of study treatment maximally 7 days after the date when the blood sample was drawn.

Visit 6 after 52 weeks is the final visit. At each visit, the same blood tests for determining Hb, TSAT and serum pregnancy are carried out with the exception of Visit 6 when only Hb and TSAT tests are performed. Further, in a subset of subjects samples for determination of blood biomarkers is retained at Weeks 6 (Visit 3), 24 (Visit 5) and 52 (Visit 6). Details concerning the blood samples storage are provided in an instruction manual and results of these exploratory analyses are not part of the clinical study report of the main study. It is planned to perform biomarker analyses in blood samples from approximately 60% of randomized subjects. The decision concerning which biomarkers will be analyzed will be made by in collaboration with the Steering Committee, together with the Sponsor. If the biomarker samples will not be analyzed, they will be destroyed. Details concerning the blood samples storage will be provided in an instruction manual. The statistical analysis for the biomarker blood samples will be detailed in a separate biomarker SAP and results of these exploratory analyses will be not part of the clinical study report of the main study.

Study treatment is prepared by an unblinded study personnel using black syringes and once prepared, study treatment is administered immediately thereafter using a curtain (or similar) to maintain subject blinding. Each subject is observed for adverse effects for at least 30 minutes following each injection of study treatment. The unblinded study personnel is not involved in any study assessments (efficacy or safety) for the subject concerned.

*Following section in italics is applicable for The Netherlands only (NL only):

The lower threshold of Hb values is set to 10 g/dL.

During Visit 2 in week 0 (baseline visit) the following additional parameters are assessed:
Completion of the self-administered KCCQ-12 and European quality of life—5 dimensions questionnaire (EQ-5D):
Kansas City Cardiomyopathy Questionnaire
Health-related quality of life (HRQoL) is assessed using the validated KCCQ-12. The KCCQ-12 is a self-administered, disease-specific instrument for measuring HRQoL in subjects with HF regardless of aetiology. It is a 12-item questionnaire that quantifies physical function, symptoms (frequency, severity and recent change), social function, self-efficacy and knowledge, and quality of life (QoL).

The EQ-5D questionnaire is a brief, utility-based generic HRQoL instrument. It consists of a health descriptive system and a visual analogue scale (VAS) for respondents to self-classify and rate their health status on the day of administration of the instrument. The descriptive system has 5 items/dimensions (i.e. mobility, self-care, usual activities, pain/discomfort and anxiety/depression). The VAS is a vertical, graduated (0-100 points) 20 cm “thermometer”, with 100 at the top representing “best imaginable health state” and 0 at the bottom representing “worst imaginable health state”. The VAS records the respondent's self-rated health on a 20 cm vertical, VAS with endpoints labelled “the best health you can imagine” and “the worst health you can imagine”. This information can be used as a quantitative measure of health as judged by the individual respondents. The subject completes the paper-based EQ-5D after the KCCQ-12 but before any other assessment or procedure for the visit concerned is performed.
Physical examination: any clinically relevant abnormalities or findings are recorded
Recording of body height and weight
Recording of vital signs. The subject's seated systolic and diastolic blood pressure in addition to pulse rate and rhythm are measured after at least 10 minutes of rest. If blood pressure is measured manually, the diastolic blood pressure must be read at the disappearance of sounds (Korotkoff Phase V). If the disappearance of sound is not detectable, Korotkoff Phase IV are used for manual measurements; care is taken not to miss a “silent gap”. Any new clinically relevant change as assessed by the Investigator, in blood pressure readings, pulse rhythm or rate are reported.
12-lead electrocardiogram (ECG)
Recording of the appropriate medical history. Information collected includes the aetiology of HF, the date when HF was first diagnosed, and other clinically relevant past and present medical conditions which have been diagnosed/occurred up to at least 12 months prior. Any medically important conditions sustained by the subject which extend beyond 12-months prior to the start of the study are also registered.
Assessment of the following symptoms and signs related to HF:
Dyspnoea on exertion,
Dyspnoea at rest while sitting,
Orthopnoea, and paroxysmal nocturnal dyspnoea.
The Investigator must assess the following clinical signs for HF:
Peripheral oedema,
Pulmonary congestion (crackles, rales),
Liver enlargement,
Presence of a third heart sound (S3 gallop),
Jugular venous distension.
Assessment of NYHA class:
NYHA functional classification is made using the classification as shown Table 5:

TABLE 5

| Class | Description |
|---|---|
| Class I | Patients have cardiac disease but without the |
| | resulting limitations of physical activity. |
| | Ordinary physical activity does not cause undue |
| | fatigue, palpitation, dyspnoea, or anginal pain. |
| Class II | Patients have cardiac disease resulting in slight |
| | limitation of physical activity. They are comfortable |
| | at rest. Ordinary physical activity results in fatigue, |
| | palpitation dyspnoea, or anginal pain. |
| Class III | Patients have cardiac disease resulting in marked |
| | limitation of physical activity. They are comfortable |
| | at rest. Less than ordinary physical activity causes |
| | fatigue, palpitation, dyspnoea, or anginal pain. |
| Class IV | Patients have cardiac disease resulting in inability |
| | to carry on any physical activity without discomfort. |
| | Symptoms of cardiac insufficiency or of the anginal |
| | syndrome may be present even at rest. If any physical |
| | activity is undertaken, discomfort is increased. |

Documentation of prior and current concomitant treatments
Final review of inclusion/exclusion criteria and confirmation of eligibility

The site contacts the subject by telephone at Week 2 (Telephone Call 1), Week 4 (Telephone Call 2), and Week 36 (Telephone Call 3), after randomization to enquire about the subject's health status and enquire if the subjects experienced a deterioration of their condition or if they were hospitalized since the last visit contact and/or if there were changes made to their concomitant treatments. The subjects are requested to complete the KCCQ-12 questionnaire at home on the same day as the Week 2, Week 4 and Week 36 telephone call. The subjects are instructed to return the completed questionnaires at the following outpatient clinic visit.

The following are tests and actions are performed during the outpatient clinic visits at Week 6 (Visit 3), Week 12 (Visit 4), Week 24 (Visit 5) and Week 52 (Visit 6) in addition to the blood tests described above:
At Week 6 (Visit 3) and Week 52 (Visit 6) only: Collection of self-administered (paper)
questionnaires (KCCQ-12 and EQ-5D) completed by the subject at Week 2, Week 4 and at Week 36, respectively.
Completion of the self-administered KCCQ—
Completion of EQ-5D at Week 6 (Visit 3), Week 24 (Visit 5) and Week 52 (Visit 6)
Assessment of the following symptoms and signs related to HF:
Dyspnoea on exertion,
Dyspnoea at rest while sitting,
Orthopnoea, and paroxysmal nocturnal dyspnoea.
The Investigator must assess the following clinical signs for HF:
Peripheral oedema,
Pulmonary congestion (crackles, rales),
Liver enlargement,
Presence of a third heart sound (S3 gallop),
Jugular venous distension.
Assessment of NYHA class: NYHA functional classification is made using the classification as shown Table 5 above.
Body weight
Vital signs: The subject's seated systolic and diastolic blood pressure in addition to pulse rate and rhythm are measured after at least 10 minutes of rest. If blood pressure is measured manually, the diastolic blood pressure is read at the disappearance of sounds (Korotkoff Phase V). If the disappearance of sound is not detectable, Korotkoff Phase IV are used for manual measurements; care is taken not to miss a “silent gap”. Any new clinically relevant change as assessed by the Investigator, in blood pressure readings, pulse rhythm or rate is reported
Documentation of changes made to/additions of concomitant treatments.
At Week 6 (Visit 3) only: Administration of the repletion dose of study treatment based on the iron need as assessed at the baseline visit. Dosing only in subjects for whom Hb ≤15 g/dL and if serum pregnancy test is negative in women of childbearing potential (see Section 9.2.3.13)
At Weeks 12 (Visit 4) and 24 (Visit 5) only: Administration of study treatment ONLY if ID persists and Hb ≥8 g/dL* and ≤15 g/dL and serum pregnancy test negative for women of childbearing potential (see Section 9.2.3.13)

*Following section in italics is applicable for The Netherlands only (NL only):

The lower threshold of Hb values is set to 10 g/dL.
Assessment of and reporting of events suggestive of the study outcomes: Reported adverse events are assessed to determine if they are suggestive of the study outcomes. This includes emergency room admissions and unscheduled outpatient clinic visits to treat worsening HF.
12-lead ECG at Week 52 (Visit 6) only
Physical examination at Week 52 (Visit 6) only: any clinically relevant abnormalities or findings are recorded.
Check for the occurrence of AEs. Document the events accordingly (see Section 10)
At Week 24 (Visit 5) only: Provision of the self-administered questionnaire (KCCQ 12) for completion at Week 36 (Telephone Call 3)
If ID persists at Week 52 (Visit 6), are treated in accordance with local routine/standard care

### Patient Inclusion Criteria

Participants in the study have to meet the following criteria:

1. Patients hospitalized at the time the study begins for an episode of acute heart failure (AHF) where AHF is the primary reason for hospitalization. All of the following (i.e., items a to d) must apply:
a. Upon admission for the AHF episode, persistent dyspnoea at rest in a recumbent sitting position(30-45°) or with minimal exertion
b. Upon or during the AHF admission, at least 2 of the following clinical findings were present:
   i. Congestion on chest X-ray
   ii. Rales on chest auscultation
   iii. Oedema ≥1+ on a 0-3+ scale, indicating indentation of skin with mild digital pressure that requires 10 or more seconds to resolve in any dependent area including extremities or sacral region
   iv. Elevated jugular venous pressure (≥8 cm H₂O)
c. Natriuretic peptide levels, measured 24 hours of the AHF admission must have been:
   i. Brain natriuretic peptide (BNP) ≥400 pg/mL or N-terminal-pro-brain natriuretic peptide (NT-proBNP) ≥1600 pg/mL or
   ii. BNP ≥600 pg/mL or NT-proBNP ≥2,400 pg/mL for subjects presenting with atrial fibrillation when the blood sample was taken
   iii. For subjects treated with an angiotensin receptor neprilysin inhibitor (ARNI) in the previous 4 weeks prior to randomization only NT proBNP values should be considered
d. AHF episode treated with minimally 40 mg of IV furosemide (or equivalent IV loop diuretic defined as 20 mg of torasemide or 1 mg of bumetanide)
   2. Subject is iron deficient defined as serum ferritin <100 ng/mL or 100 ng/mL ≤serum ferritin ≤299 ng/mL if TSAT <20%.
   3. Left ventricular ejection fraction <50% (assessed and documented within 12 months prior to randomization). The LVEF may be measured by echocardiography, computerized tomography (CT scan), magnetic resonance imaging (MRI) or ventricular gated single-photon emission computed tomography (SPECT) or radionuclide angiography (MUGA).
   4. Male or female aged ≥18 years old.
   5. Subject (or legally acceptable representative)* has provided the appropriate written informed consent. Subject must provide written informed consent before any study-specific procedures are performed.

* Following section in italics is applicable for The Netherlands only (NL only):

The option that legally accepted representatives of subjects can sign the written informed consent is not valid for sites in The Netherlands.

### Patient Exclusion Criteria

Participants meeting the following criteria are not included in the study:
1. Dyspnoea due to non-cardiac causes such as acute or chronic respiratory disorders or infections (i.e., severe chronic obstructive pulmonary disease, acute bronchitis, pneumonia, primary pulmonary hypertension).
2. Temperature >38° C. (oral or equivalent), active infective endocarditis, sepsis, systemic inflammatory response syndrome, or any other active infection requiring anti-microbial treatment at any time during an Index hospitalization.
3. Documented restricted amyloid myocardiopathy, or acute myocarditis or hypertrophic obstructive, restrictive, or constrictive cardiomyopathy. (Note that it does NOT include restrictive mitral filling patterns seen on Doppler echocardiographic assessments of diastolic function).
4. Clinical evidence of acute coronary syndrome, transient ischemic attack or stroke, within the last 30 days prior randomization.
5. Severe valvular or left ventricular outflow obstruction disease needing intervention.
6. Coronary-artery bypass graft, cardiac resynchronization therapy device implantation, percutaneous intervention (e.g., cardiac, cerebrovascular, aortic, diagnostic catheters are allowed) or major surgery that led to significant blood loss, including thoracic and cardiac surgery, within the last 3 months prior to randomization.
7. Subject has a body weight <35 kg at randomization.
8. Subject at an immediate need of transfusion or with a Hb <8 g/dL* or with a Hb >15 g/dL.
9. Subjects on treatment for Vitamin B12 and/or serum folate deficiency. Note: Use of Vitamin B12 and folic acid as supplement therapy (not for deficiency treatment) is permitted.
10. Subject with a known anaemia not attributed to ID (e.g., other microcytic anaemia) or with an evidence of iron overload (e.g., haemochromatosis) or disturbances in the utilization of iron.
11. Subject has known hypersensitivity to any of the study products to be administered or known serious hypersensitivity to other parenteral iron products.
12. Subject with known severe allergies including drug allergies, history of severe asthma, eczema or other atopic allergy and in subjects with immune or inflammatory conditions (e.g., systemic lupus erythematosus, rheumatoid arthritis).
13. History of erythropoietin stimulating agent, IV iron therapy, and/or blood transfusion in previous 3 months prior to randomization.
14. Oral iron therapy at doses >100 mg/day in previous 4 weeks prior to randomization. Note: Ongoing use of multivitamins containing iron <75 mg/day are permitted.
15. Currently receiving systemic chemotherapy and/or radiotherapy.
16. Renal dialysis (previous, current or planned within the next 6 months).
17. Subject has known history of malignancy of any organ system (except non-invasive basal cell carcinoma, squamous cell carcinoma of the skin or cervical intra-epithelial neoplasia) within the last 5 years.
18. Terminal illness other than HF with expected survival <12 months.
19. Chronic liver disease (including active hepatitis) and/or alanine transaminase or aspartate transaminase above 3 times the upper limit of the normal range.
20. Subjects with known hepatitis B surface antigen positivity and/or hepatitis C virus ribonucleic acid positivity.
21. Subject previously randomized into this study. Note: Subjects may be rescreened but when rescreened, all tests must fall inside the maximum specified screening windows for each criterion.
22. Subject is currently enrolled in or has completed any other investigational device or drug study <30 days prior to screening, or is receiving other investigational agent(s).
23. Subject is pregnant (e.g., positive human chorionic gonadotropin test) or breast feeding.
24. If of childbearing potential, subject is not using adequate contraceptive precautions. Subject must agree to use adequate contraception during the study and for 1 month after the last dose of study treatment. A highly effective method of birth control must be used.
25. Subject has a history of drug or alcohol abuse within 2 years prior to screening.
26. Subject has a significant medical condition(s), anticipated need for major surgery during the study, or any other kind of disorder that may be associated with increased risk to the subject, or may interfere with study assessments, outcomes, or the ability to provide written informed consent or comply with study procedures, in the Investigator's opinion.

* Following section in italics is applicable for The Netherlands only (NL only):

The lower threshold of Hb values is set to 10 g/dL.

### Study Endpoints

The primary endpoints investigated are, relative to placebo, the evaluation of the effect of IV FCM on repeated HF hospitalizations and CV death up to 52 weeks after randomization.

The secondary endpoints evaluate, relative to placebo, the effect of IV FCM on:
The composite of recurrent CV hospitalizations and CV death up to 52 weeks after randomization.
HF hospitalizations up to 52 weeks after randomization (analyzed as recurrent event).
CV mortality analyzed as time to first event at 52 weeks after randomization.
The composite of HF hospitalizations or CV death analyzed as time to first event at 52 weeks after randomization.
Days lost due to HF hospitalizations or CV death at 52 weeks after randomization.

The other endpoints evaluate, relative to placebo, the effect of IV FCM on:
The composite of recurrent HF hospitalizations and CV death up to 30 days after randomization.
The composite of recurrent CV hospitalizations and CV death up to 30 days after randomization.
The composite of HF hospitalizations or CV death analyzed as time to first event at 30 days after randomization.
The composite of CV hospitalizations or CV death analyzed as time to first event at 30 days after randomization.
HF hospitalizations up to 30 days after randomization (analyzed as recurrent event).
HF hospitalizations up to 30 days and 52 weeks after randomization (analyzed as time to first event).
CV hospitalizations up to 30 days and 52 weeks after randomization (analyzed as recurrent event and time to first event).
The composite of CV hospitalizations or CV death analyzed as time to first event at 30 days and 52 weeks after randomization.
Cardiovascular mortality analyzed as time to first event at 30 days after randomization. All-cause mortality analyzed as time to first event at 30 days and 52 weeks after randomization.
Proportion of patients with an event (HF hospitalizations, CV hospitalizations, CV mortality, composite and individual categories).
Change from baseline in NYHA functional class as assessed at 6, 12, 24 and 52 weeks after randomization.
Change from baseline in the Kansas City Cardiomyopathy Questionnaire-12 up to 52 weeks after randomization.
Change from baseline in the European quality of life—5 dimensions questionnaire up to 52 weeks after randomization.
Days lost due to HF hospitalizations or CV death at 30 days after randomization.

### Statistical Methods

All statistical analyses are performed using SAS Version 9.3 or later (SAS Institute Inc. SAS/STAT, Cary, N.C., United States). The level of significance to be used will be 0.05. Detailed methodology for summary and statistical analyses of the data collected in this trial will be documented in a Statistical Analysis Plan (SAP), which will be finalized prior to unlocking of the study data base. A general description of the planned methods is provided below. Any deviation from the SAP will be noted and explained in the final study report.

### Sample Size and Power Calculations

The recurrent HF hospitalization and CV death rates in the control group is extrapolated as 0.9 events/year using the data from the EVEREST study and as 0.61 events/year using the data from the ESC-HF study. For this study, it is anticipated that approximately 35% of subjects will sustain either a CV death or will sustain at least one HF hospitalization. It is also anticipated that 12% of subjects will sustain a CV death. Assuming that 50% of subjects who sustain a CV death will not undergo any prior HF hospitalization and that the average number of HF hospitalizations per subject with at least one HF hospitalization will be estimated as 2, the number of events per 100 years has been estimated as follows: 12 CV deaths+2*(35-6) HF hospitalizations, which equates to approximately 70 events/100 years of follow-up for HF hospitalizations and CV death.

Concerning the event rate ratio, it is assumed that there will be a 30% reduction in HF hospitalizations for subjects allocated to FCM and that CV death rates will be similar between the FCM and placebo groups. It is therefore assumed that the rate ratio between FCM and placebo for the composite of recurrent HF hospitalizations and CV deaths will be approximately 25%.

The dispersion factor used in negative binomial regression is a measure of the mean-variance. There is currently insufficient data to estimate the negative binomial dispersion. For this sample size calculation, a dispersion factor (K) of 1 was assumed.

The sample size calculation is done in the software NCSS PASS-14 [39] using the sample size formula proposed by Zhu and Lakkis 2014 [40] to compare two negative binomial rates.

Assuming a recurrent HF hospitalization and CV death of 0.7 events/year the placebo group, in total, 1,000 subjects (500 per study treatment group) would be required to demonstrate a statistically significant rate ratio of 0.75 (i.e., 25% reduction of recurrent events between the FCM and placebo groups) with a power of 80% and a 2-sided alpha of 0.05. Taking into account 9% loss to follow-up, a sample size of 1,100 subjects (550 per treatment group) is planned.

### Full Analysis Set

The full analysis set (FAS) consists of all subjects who satisfy the following criteria:
Randomised to either of the two treatment groups
The administration of study treatment was started

The FAS data set is analyzed based on the randomized treatment arm.

### Per-Protocol Set

The per-protocol (PP) analysis set consists of all subjects who, in addition to the FAS criteria, have had no major protocol violations.

### Safety Set

The safety set consists of all randomized subjects administered at least 1 dose of study treatment. The subjects in this group will be analyzed based on the treatment received.

### Background and Demographic Characteristics

The baseline and demographic characteristics is summarized per treatment group.

### Study Treatment

The total amount of study treatment given is calculated for each subject and is compared to the amount expected to be given for each subject. Treatment compliance is calculated for each subject and summarized by treatment group.

### Concomitant Therapy

Concomitant medications is categorized according to a standard dictionary (World Health Organization Drug Classification). Counts and percentages of subject use for each medication are computed and summarized by treatment group.

### Efficacy Evaluations of Endpoints

The HF hospitalization and CV death rates per 100 patient-years of follow-up as adjudicated by the Clinical Endpoint Committee (CEC), is calculated by dividing the total number of HF hospitalizations, and CV deaths by the total follow-up duration of all subjects in each group. The rate ratio (95% CI and p-value) for this analysis will be analyzed using a negative binomial model. Compared to the Poisson distribution, the negative binomial distribution allows for different individual tendencies (frailties) with respect to their risks of repeat hospitalizations.

The negative binomial model is adjusted for the following baseline covariates: sex, age, HF aetiology (ischemic/non-ischemic), HF duration (newly diagnosed at Index hospitalization/known documented HF prior to Index hospitalization) and country. A sensitivity analyses is performed using an unadjusted model.

The primary outcome analysis is performed on the FAS and uses the CEC adjudicated events. A sensitivity analysis is performed on the PP population set.

Descriptive statistics provide, per treatment group, the total number of events and the number (%) of subjects with at least one event.

A graphical representation of the estimated cumulative hazard rate is also provided. As the analysis of HF hospitalizations could be confounded by the competing risk of death, a confirmatory analysis will be performed on the FAS population set using the joint frailty model in order to analyze repeat hospitalization rate whilst accounting for their associated mortality rate.

The analyses of secondary and other endpoints are performed on the FAS and used the CEC adjudicated events for hospitalizations and mortality-related outcomes. For the recurrent event analysis, the same analysis as that described for the primary outcome is performed. For the time to first event analysis, the incidence of events are documented by treatment group with the total number of events, the number of subjects with at least 1 event and the event hazard rate per 100 patient years “at risk” (estimated as the number of patients with at least 1 event divided by the patient years at risk of event). Patient years at risk of event are taken as the sum of the observation time from start of study treatment until the first occurrence of the event concerned, or until censoring. The hazard ratio (relative to placebo), its 95% CI and the p-value test is provided using Cox regression. The proportion of patients with an event (HF hospitalizations, CV hospitalizations, CV mortality; composite and individual categories) were also reported.

The change in NYHA class were analyzed using a repeated measurement analysis of the ordered polytomous regression adjusted for treatment, time and the baseline NYHA value.

Quality of Life is assessed using the KCCQ-12 and EQ-5D. The analysis of treatment difference on the KCCQ-12 score at Week 2, Week 4, Week 6, Week 12, Week 24, Week 36 and Week 52 is done by comparing the model adjusted means of the corresponding visit based on a model for repeated-measures including terms for treatment, baseline, time and treatment-by-time with an unstructured covariance matrix to model the within-patient variability. The analysis of treatment difference on the EQ-5D score is done in the same manner for the data collected at Week 6, Week 24 and Week 52.

### Heart-FID Study

The HEART-FID study is a randomized, double-blind, placebo-controlled, prospective, multi-centre study to investigate the efficacy and safety as well as the effect om functional capacity of intravenous (IV) ferric carboxymaltose (FCM), relative to placebo, in the treatment of over 3000 participants in heart failure with a reduced ejection fraction and with iron deficiency. The study assesses the effects of IV FCM on the 12-month rate of death, hospitalization for worsening heart failure, and the 6-month change in 6 minute walk test (6MWT) for patients in heart failure with iron deficiency.

### Treatment

After an initial screening period of up to 28 days, eligible participants at 200 study sites are stratified by region and randomized in a 1:1 ratio to FCM or placebo for treatment.

Study drug administration occurs on Day 0 (date of randomization) and Day 7 (±2) as an undiluted slow IV push. Group A (FCM) receives a 750 mg undiluted blinded dose of IV FCM at the rate of approximately 100 mg (2 mL)/minute; Group B (placebo) receives a blinded placebo (15 mL of normal saline) IV push at 2 mL/minute. Participants in Group A with body weight <50 kg (110 pounds) have individual FCM doses adjusted to 15 mg/kg, not to exceed an individual dose of 750 mg, or a cumulative dose of 1500 mg per treatment cycle. All participants are dosed every 6 months for the duration of the trial as applicable. All participants are dosed every 180 and 187 days from their previous dose for the entire study duration. Randomized treatment is administered if hemoglobin <13.5 g/dl (females) or <15.0 g/dl (males) and serum ferritin <100 ng/ml or 100 to 300 ng/mL with TSAT <20%. Additional study visits occur at 3 month intervals.

Participants not meeting post-randomization lab criteria for blood counts and iron studies and all participants randomized to the placebo arm are administered IV placebo at each visit.

In a subset of sites, all participants return for recurrent laboratory assessment (chemistry, hematology and iron indices) at Day 21 (±7) after each course of investigational treatment. For all participants, hematology, ferritin, and transferrin saturation (TSAT), with appropriate safety evaluations, to determine additional treatment, occur at 6 month intervals. The post-randomization phase is variable with a minimum of 365 days.

Unblinded site personnel, responsible for preparation and administration of the FCM or placebo, ensure that the participant and all blinded site staff are not able to observe the preparation or administration of study treatment.

The study drug provided by Luitpold Pharmaceuticals, Inc. has the trade name Injectafer® and is supplied as 15 ml vials, containing 750 mg of iron as 5% w/v iron containing a polynuclear iron(III)-hydroxide 4(R)-(poly-(1-->4)-O α-D-glucopyranosyl)oxy-2 (R), 3(S), 5(R), 6-tetrahydroxy-hexonate complex in a solution of water for injection [50 mg/ml] and is labeled according to FDA investigational regulatory requirements. Placebo (normal saline) is supplied as 15 ml vials.

### Inclusion Criteria for Participants

1. Adult (≥18 years of age) able to provide informed consent.
2. Stable heart failure (NYHA II-IV) on maximally-tolerated background therapy (as determined by the site Principle Investigator) for at least 4 weeks with no dose changes in heart failure drugs during the last 2 weeks.
3. Able and willing to perform a 6MWT at the time of randomization.
4. Reduced left ventricular ejection fraction. Assessment was performed at least 12 weeks after major cardiac surgical intervention including coronary artery bypass graft (CABG), valvular repair/replacement, or cardiac resynchronization therapy (CRT) device implantation.
   a. Left ventricular ejection fraction 35% obtained during the screening visit OR either of the following
      i. Historical value of ejection fraction 35% within 12 months of screening visit
      ii. Historical value of ejection fraction 25% within 24 months of screening visit
5. Hemoglobin >9.0 g/dL and <13.5 g/dL (females) or <15.0 g/dL (males).
6. Serum ferritin <100 ng/mL or 100 to 300 ng/mL with TSAT <20%. 7. Either documented hospitalization for heart failure within 12 months of enrollment or screening visit N-terminal-pro-brain natriuretic peptide (NT-proBNP) >600 pg/mL (or BNP >200 pg/mL) for patients with normal sinus rhythm or NT-proBNP >1000 pg/mL (or BNP >400 pg/mL) for patients with atrial fibrillation. NOTE: NT-proBNP was used to confirm eligibility for patients taking sacubitril/valsartan.

### Exclusion Criteria for Participants

1. Current or planned oral iron supplementation. Iron-containing multivitamins (<30 mgs/day) are permitted.
2. Known hypersensitivity reaction to any component of FCM.
3. History of acquired iron overload, or the recent receipt (within 3 months) of erythropoietin stimulating agent, IV iron therapy, or blood transfusion.
4. Acute myocardial infarction, acute coronary syndrome, transient ischemic attack, or stroke within 3 months of enrollment.
5. Uncorrected severe aortic stenos is, severe valvular regurgitation, or left ventricular outflow obstruction requiring intervention.
6. Current atrial fibrillation or atrial flutter with a mean ventricular response rate >100 per minute (at rest).
7. Current or planned mechanical circulatory support or heart transplantation.
8. Hemodialysis or peritoneal dialysis (current or planned within the next 6 months).
9. Documented liver disease, or active hepatitis (i.e. alanine transaminase or aspartate transaminase >3 times the upper limit of normal range).
10. Current or recent (within 3 years) malignancy with exception of basal cell carcinoma or squamous cell carcinoma of the skin, or cervical intraepithelial neoplasia.
11. Known gastrointestinal bleeding. Patients with screening ferritin <15 ng/ml must have an appropriate evaluation within 3 months of screening.
12. Female participant of child-bearing potential who is pregnant, lactating, or not willing to use adequate contraceptive precautions during the study and for up to 5 days after the last scheduled dose of study medication.
13. Inability to return for follow up visits within the necessary windows.

### Screening Visit

The screening phase lasts from day −28 to day 0.

Each participant who has signed the informed consent and has qualified for inclusion undergoes the following clinical evaluations to confirm eligibility for the study (all procedures to be performed by blinded study personnel):
Demographic and medical history including NYHA heart failure class and prior heart failure hospitalizations
Left ventricular ejection fraction (historical values are to be used if performed within 12 months of the screening visit, or 24 months if LVEF 25%) is performed at least 12 weeks after major cardiac intervention-including coronary artery bypass grafting (CABG), valvular intervention, or cardiac resynchronization therapy device implantation
Blood samples for hematology, iron indices, and NT-proBNP (local laboratory)
Concomitant medications
Review of inclusion/exclusion criteria

### Treatment Phase

### Day 0 Visit

All eligible participants are randomized to either Group A or Group B in a 1:1 ratio based on a pre-determined randomization schedule via an interactive response technology (IRT) system.

The following are obtained and/or completed before contacting IRT for randomization: For all participants (all procedures performed by blinded study personnel):
Verification of all inclusion and exclusion criteria
Height and weight
Targeted physical exam
Blood samples for central lab hematology, chemistries and iron indices for all participants; Vitamin D, PTH for participants at sites selected for post-dose chemistry follow-up visits.
Review of concomitant medications
Urine pregnancy test (women of childbearing potential only)
Administration of 6MWT per standardized procedure

### Group A:

Verification of amount of single FCM dose (15 mg/kg up to a maximum dose of 750 mg) (unblinded staff)
Pre-administration, measurements of heart rate, blood pressure, and body temperature (blinded staff)
Administration of FCM as a slow IV injection at the rate of approximately 2 mL/minute taking appropriate measures to ensure the participant and all blinded staff members remain blinded to the treatment being administered (unblinded staff)
Documentation of start and stop time of FCM administration and the total dose and volume administered (unblinded staff)
Post-administration, measurement of heart rate and blood pressure immediately after and 30 minutes after FCM administration (blinded staff)
Adverse event/serious adverse event assessment (starting at beginning of FCM injection) (blinded staff)

### Group B:

Pre-administration, measurements of heart rate, blood pressure, and body temperature (blinded staff)
Administration of a 15 mL dose of placebo (normal saline) as a slow IV injection at the rate of approximately 2 mL/minute taking appropriate measures to ensure the participant and all blinded staff members remained blinded to the treatment being administered (unblinded staff).
Documentation of start and stop time of placebo administration and the total volume administered (unblinded staff).
Post-administration, measurements of heart rate and blood pressure immediately after and 30 minutes after placebo administration (blinded staff)
Adverse event and serious adverse event assessment (starting at beginning of placebo injection) (blinded staff)

### Day 7 Visit

All participants return to the clinic for study drug dosing on Day 7(±2). Prior to the administration of the study drug, the participant is evaluated clinically to assess the development of clinically significant conditions that may contraindicate dosing.

### Group A

Verification of amount of single FCM dose (15 mg/kg up to a maximum dose of 750 mg) (unblinded staff)
Pre-administration, measurements of heart rate, blood pressure, and body temperature (blinded staff)
Administration of FCM as a slow IV injection at the rate of approximately 2 mL/minute taking appropriate measures to ensure the participant and all blinded staff members remain blinded to the treatment being administered (unblinded staff).
Documentation of start and stop time of FCM administration and the total dose and volume administered (unblinded staff)
Post-administration, measurements of heart rate and blood pressure immediately after and 30 minutes after FCM administration (blinded staff)
Adverse event/serious adverse event assessment, including evaluation of potential endpoint events (blinded staff)
Review of concomitant medications

### Group B

Pre-administration, measurements of heart rate, blood pressure, and body temperature (blinded staff)
Administration of a 15 mL dose of placebo (normal saline) as a slow IV injection at the rate of approximately 2 mL/minute taking appropriate measures to ensure the participant and all blinded staff members remain blinded to the treatment being administered (unblinded staff).
Documentation of start and stop time of placebo administration and the total volume administered (unblinded staff)
Post-administration, measurements of heart rate and blood pressure immediately after and 30 minutes after placebo administration (blinded staff)
Adverse event/serious adverse event assessment, including evaluation of potential endpoint events (blinded staff)
Review of concomitant medications (blinded staff)

### Chemistry Laboratory Collection Subset −Day 21 (±7)

In a subset of sites approximately 500 participants, have central lab clinical laboratories (chemistry, hematology and iron indices) collected following the initial and each subsequent course (approximately every 6 months) of study drug treatment (FCM or Placebo). The participants have chemistry laboratories collected 21±7 days post the first treatment for that course (i.e. Study Days 21±7, 201±7, 381±7, 561±7, 741±7, 921±7 . . . End of Study). (blinded staff).

### 90 Day Follow-Up

Following the initial and all subsequent courses of study drug treatments each participant is contacted in person or via telephone 90±14 days post the first treatment for that course (i.e. study Days 90±14, 270±14, 450±14, 630±14, 810±14, 990±14 . . . End of Study) During these visits the following is performed:
Adverse event/serious adverse event assessment, including evaluation of potential endpoint events (blinded staff).

### 6 Month Laboratory Evaluation

Participants receive an additional course of study medication every 180 (±7) days. Within 2 to 20 days prior to these scheduled dosing visits, all participants return to the clinic to obtain central lab hematology, chemistry, and iron indices laboratory tests. (Blood collected by blinded staff)

### Additional Study Drug Dosing (Every 6 Months)

All participants are dosed every 6 months. At each 6-month interval, a course of 2 doses of study drug is administered as described above for Day 0 and Day 7. For group A, FCM is administered if Hb <13.5 g/dL (females) or <15.0 g/dL (males) and serum ferritin <100 ng/mL or 100 to 300 ng/mL with TSAT <20%; placebo (normal saline) is administered to participants in the FCM group who do not meet the above criteria. All group B participants receive placebo (normal saline).

### 6 Month Dosing Visit #1 (Days 180±7, 360±7, 540±7, 660±7, 840±7, 1,020±7 . . . End of Study)

On the first of the two dosing visits, the following is performed by blinded study staff for all participants:
   Weight
   Urine pregnancy test (women of childbearing potential only)
   Adverse event/serious adverse event assessment, including evaluation of potential endpoint events.
   Review of concomitant medications
   Administration of 6MWT per standardized procedure (at the 6 and 12 month visits).

For Group A participants the following is performed:

Verification if participant will receive FCM or placebo, based on the following criteria from recent labs (within 20 days). Participants receive FCM if the Hb <13.5 g/dL (females) or <15.0 g/dL (males) and serum ferritin <100 ng/mL or 100 to 300 ng/mL with TSAT <20%. If the participant does not meet these criteria, placebo (normal saline) is administered. (unblinded staff)
As appropriate based on criteria above, verification of amount of single FCM dose (15 mg/kg up to a maximum dose of 750 mg) or placebo (15 mL). (unblinded staff)
Pre-administration, measurements of heart rate, blood pressure, and body temperature. (blinded staff)
Administration of FCM or placebo as a slow IV injection at the rate of approximately 2 mL/minute. Appropriate measures are taken to ensure the participant and all blinded staff members remain blinded to the treatment being administered (unblinded staff)
Documentation of start and stop time of IV administration, and the total dose and volume administered (unblinded staff)
Post-administration of FCM, measurements of heart rate and blood pressure immediately after and 30 minutes after FCM administration (blinded staff)

For Group B participants the following was performed:
Pre-administration, measurements of heart rate, blood pressure and body temperature. (blinded staff)
Administration of a 15 mL dose of placebo (normal saline) as a slow IV injection, at the rate of approximately 2 mL/minute. Taking appropriate measures to ensure the participant and all blinded staff members remain blinded to the treatment being administered (unblinded staff)
Documentation of start and stop time of placebo administration and the total volume administered. (unblinded staff)
Post-administration of placebo, measurements of heart rate and blood pressure immediately after and 30 minutes after placebo administration (blinded staff).

### Six Month Dosing Visit #2 (Days 187±7, 367±7, 547±7, 667±7, 847±7, 1,027±7 . . . End of Study)

The second of the 2 dosing visits occurs at Day 7 (±2) after the first, with the following performed for all participants:
Adverse event/serious adverse event assessment, including evaluation of potential endpoint events (blinded staff), and review of concomitant medications (blinded staff).

For Group A participants the following is performed:
Verification of amount of single FCM dose (15 mg/kg up to a maximum dose of 750 mg) or placebo (15 mL). Participants receive the same product (FCM or placebo) as received at the first dose of this course of treatment. (unblinded staff)
Pre-administration, measurements of heart rate, blood pressure, and body temperature. (blinded staff)
Administration of FCM or placebo as a slow IV injection at the rate of approximately 2 mL/minute. Taking appropriate measures to ensure the participant and all blinded staff members remain blinded to the treatment being administered (unblinded staff).
Documentation of start and stop time of IV administration and the total dose and volume administered. (unblinded staff)
Post-administration, obtain heart rate and blood pressure immediately after and 30 minutes after FCM administration. (blinded staff)

For Group B participants the following is performed:
Pre-administration, measurements of heart rate, blood pressure, and body temperature. (blinded staff).
Administration of a 15 mL dose of placebo (normal saline) as a slow IV injection at the rate of approximately 2 mL/minute. Taking appropriate measures to ensure the participants and all blinded staff members remain blinded to the treatment being administered (unblinded staff)
Documentation of start and stop time of placebo administration and the total volume administered. (unblinded staff)
Post-administration, measurements of heart rate and blood pressure immediately after and 30 minutes after placebo administration. (blinded staff).

### End of Study Visit

End of study visits for all participants are scheduled once the last participant has reached 12 months on study and at least 771 participants have experienced an event of cardiovascular death or hospitalization for heart failure. When possible, the participants return to the clinic and the following is performed by blinded study staff:
Targeted physical exam
Vital signs including blood pressure and heart rate
Blood samples for central lab hematology, chemistries, iron indices and NT-proBNP.
Urine pregnancy test (women of childbearing potential only)
Review of concomitant medications
Adverse event/serious adverse event assessment, including evaluation of potential endpoint events.

### Laboratory Assessments

Serum samples for laboratory analyses are obtained at all appropriate visits. Screening laboratory values are analyzed locally. All other visit laboratory samples are analyzed by a central clinical laboratory. All laboratory testing is provided to the investigator or his/her medically qualified designee for review and assessment. Post-dose iron indices and serum phosphorus results are provided to the designated unblinded investigator for assessment. The laboratory assessments are determined as listed in Table 6:

Hematology: Hemoglobin (Hb), hematocrit (Hct), red blood cell (RBC) count, white blood cell (WBC) count, mean corpuscular volume (MCV), mean corpuscular hemoglobin (MCH), mean corpuscular hemoglobin concentration (MCHC), red cell distribution width (RDVV), platelets, differential count, and reticulocyte count.

Chemistry: Sodium, potassium, chloride, blood urea nitrogen (BUN), creatinine, albumin, alkaline phosphatase, total bilirubin, gamma-glutamyl transferase (GGT), Aspartate aminotransferase (AST), Alanine Aminotransferase (ALT), Lactic dehydrogenase (LDH), calcium, phosphorus, glucose, bicarbonate, and magnesium.

Iron indices: Serum iron, serum ferritin, total iron binding capacity (TIBC), and percentage serum transferrin saturation (TSAT)

Other: Vitamin D, Parathyroid Hormone, NT-proBNP.

TABLE 6

| | | | | Follow-up Phase | | | | | | |
|---|---|---|---|---|---|---|---|---|---|---|
| | | | | Select | | | | | Select | |
| | | Treatment | | Sites | | | | | Sites | |
| Study Procedures | Screening | Phase | | only | | | | | only | |
| Days | −28 to −1 | 0 | 7 ± 2 | 21 ± 7^{a,k} | 90 ± 14^{a,b,l} | 160-178^{a,b} | 180 ± 7^{a,b} | 187 ± 7^{a,b} | 201 ± 7^{a,k} | EOS^{e} |
| Informed consent | X | | | | | | | | | |
| Inclusion/exclusion criteria | X | X^{j} | | | | | | | | |
| Demographics | X | | | | | | | | | |
| Targeted medical history | X | | | | | | | | | |
| Targeted Physical Exam | | X^{j} | | | | | | | | X |
| Vital signs | | X^{d} | X^{d} | | | | X^{d} | X^{d} | | X |
| Height (cm) & weight (kg)^{e} | | X^{j} | | | | | X | | | |
| Urine pregnancy test^{f} | | X^{j} | | | | | X | | | X |
| Vitamin D and PTH^{k} | | X | | | | | | | | |
| Left ventricular ejection fraction | X^{g} | | | | | | | | | |
| Randomizationⁿ | | X | | | | | | | | |
| Hematology laboratory^{h} | X | X | | X | | X | | | X | X |
| Chemistry laboratory^{h} | | X | | X | | X | | | X | X |
| Iron indices^{h} | X | X | | X | | X | | | X | X |
| 6 Minute Walk Test | | X^{j} | | | | | X^{m} | | | |
| NT-proBNP^{h} | X | X | | | | | X | | | X |
| Serious Adverse Event reporting | | X | X | | X | X | X | X | | X |
| Concomitant medications | X | X | X | | | | X | X | | X |
| IV FCM/IV Placeboⁿ | | X | X | | | | Xⁱ | Xⁱ | | |

### Study Endpoints

The primary endpoint is hierarchical composite of 1) death, 2) hospitalization for heart failure or 3) change in 6MWT. Death and hospitalizations for heart failure are evaluated at one year, change in 6MWT is evaluated at 6 months) and tested using the nonparametric Wilcoxon-type test.

The secondary endpoints are
1. Time to first event of the composite of cardiovascular death or heart failure hospitalization. The composite endpoint is composed of adjudicated occurrence of one of the following:
   a. Cardiovascular Death
      i. Death due to Heart Failure
      ii. Death due to Acute Myocardial Infarction
      iii. Sudden Cardiac Death
      iv. Death due to Stroke
      v. Death due to other Cardiovascular Causes
   b. Hospitalization for Worsening Heart Failure
2. Mean change in 6MWT from baseline to 12 months
3. Time to first event of the composite of cardiovascular death or intervention for worsening heart failure (hospitalization or urgent heart failure visits)
4. Time to first event of the composite of cardiovascular death and cardiovascular hospitalizations
5. Time to cardiovascular death

Additional events that are adjudicated for analysis of the secondary endpoints include:

a) Non-cardiovascular death
b) Hospitalization for myocardial infarction
c) Hospitalization for stroke
d) Other cardiovascular hospitalizations
e) Urgent heart failure visits

### Primary Comparison

Each participant from the treatment arm is ranked/compared with each participant from the control arm based on the 12-month experience for Death and Hospitalizations for heart failure and 6 month results for change in 6MWT to determine treatment response per the following hierarchy:

### 1. Death

If both die, the one who survives longer is better off;
If one dies and one does not, the one that survives is better off;
If neither dies, examine hospitalizations for heart failure.
2. Hospitalizations for heart failure
The one with fewer hospitalizations is better off;
If neither has been hospitalized for heart failure or the number heart failure hospitalizations is equal, compare change in 6MWT.

### 3. Change in 6MWT

The one with higher change in 6MWT is better off;

### Statistics

All statistical tests are two-tailed. Type I error of 0.05 is assumed unless otherwise specified. No adjustments for multiple testing are made.

### Stratification/Randomization

Participants who meet the inclusion/exclusion criteria are randomized in a 1:1 ratio on Day 0 to FCM or Placebo with stratification by region.

### Analysis Populations

The Intent-To-Treat (ITT) Population consists of all participants randomized to a treatment group in the study regardless of compliance with the study medication. For all analyzed using the ITT population, participants are analyzed as randomized. This is the primary population of all efficacy analyses. The Per-Protocol Population is a subset of the ITT population excluding participants who complied with the randomized treatment for less than 50% of the follow-up. In cases of medication error, treatment assignments in the per-protocol analysis are analyzed according to the actual treatment received.

### Disposition and Baseline Characteristics

The number and percent of participants who are randomized, are treated with randomized therapy, discontinue prematurely, and complete the study are summarized. The number and percent of participants are summarized for each reason for premature discontinuation.

Categorical baseline characteristics (e.g., sex and race) are summarized with the number and percent of participants in each treatment group with the characteristic. Quantitative characteristics (e.g., age and weight) are summarized with the mean, median, standard deviation, minimum value, and maximum value. Baseline characteristics are summarized for the safety and ITT populations.

### Adverse Events

Any untoward medical event experienced by a participant during the course of this clinical trial, whether or not it is related to the investigational product, at any dose, is considered an adverse event (AE).

For any laboratory abnormality, the investigator, or his/her medically qualified designee, makes a judgment as to its clinical significance. If the laboratory value is outside the normal limits and is felt to represent a clinically significant worsening from the baseline value, it is considered an adverse event. If the laboratory value is outside the normal range, but not an adverse event, the investigator comments on the findings (i.e. “not clinically significant” or “unchanged from baseline”) in the source documentation [laboratory report].

The investigator uses Table 7 to assign the adverse event severity grade.

TABLE 7

| Grade | Adjective | Description |
|---|---|---|
| 1 | Mild | Does not interfere with the |
| | | participant's usual function |
| 2 | Moderate | Interferes to some extent with |
| | | participant's usual function |
| 3 | Severe | Interferes significantly with |
| | | participant's usual function |
| 4 | Life-threating | Results in a threat to life or |
| | | in an incapacitating disability |
| 5 | Death | Results in Death |

### Timing:

Adverse events and serious adverse events are reported, from the time of randomization through the end of study. Adverse events for participants randomized and who terminate early are reported for 30 days after the last treatment.

### Relationship (Causality):

The Investigator is asked to document his/her opinion of the relationship of the event to the study drug* as follows:
NONE There is no evidence of any causal relationship.
UNLIKELY There is little evidence to suggest there is a causal relationship. There is another reasonable explanation for the event (e.g., the participant's clinical condition, other concomitant treatments).
POSSIBLE There is some evidence to suggest a causal relationship (i.e. there is a reasonable possibility that the adverse experience may have been caused by the agent). However, the influence of other factors may have contributed to the event (e.g., the participant's clinical condition, other concomitant events).
PROBABLE There is evidence to suggest a causal relationship, and the influence of other factors is unlikely.
For the purposes of this trial, “study drug” is defined as: FCM OR Placebo

### Reporting of Adverse Events (AE)

For the purposes of this study, any AE that does not meet the protocol definition of a serious AE is considered non-serious. Non-serious AEs are not collected for this trial, except for AEs leading to cessation of study medication. Disease progression is considered as a worsening of a patient's clinical condition attributable to the disease in the patient population for which the study medication is studied. It may be an increase in the severity of the disease under study, and/or increases in the symptoms of the disease. The development of the following cardiovascular disease events is recorded, however, they are considered as disease progression and are not reported as an AE/serious adverse event (SAE) during the study unless determined to be clinical endpoints. These include the events listed below, “Reporting of Events that May Require Adjudication.” Adverse experiences are elicited by nonspecific questions such as “Have you noticed any problems? Participants are encouraged to report adverse events at their onset.

### Serious Adverse Events

Definition: An adverse event is classified as SERIOUS if it met any one of the following criteria:
Death
Life-Threatening: The participant is at substantial risk of dying at the time of the adverse event or it is suspected that the use or continued use of the product will result in the participant's death.
Hospitalization (initial or prolonged): Required admission to the hospital or prolongation of a hospital stay except events which are components of the primary or secondary endpoints which will be adjudicated by the CEC Committee as noted below.
Disability: Resulted in a significant, persistent, or permanent change, impairment, damage or disruption in the participant's body function/structure, physical activities or quality of life.
Congenital Anomaly/Birth Defect.
Important medical events: Other medically important events that, in the opinion of the investigator, may jeopardize the participant or may require intervention to prevent one of the other outcomes listed above.

Suspected clinical endpoint events that may traditionally meet the definition of an SAE, are not reported by the sites in this trial as an SAE.

Reporting of Suspected Study Endpoint Events that May Require Adjudication

The following events, which are the components of the primary or secondary endpoints are adjudicated by the Clinical Events Classification (CBC) Committee of the Duke Clinical Research Institute (DCRI) for both FCM and Placebo and do not require reporting to the sponsor as an SAE:
Cardiovascular Death including:
Death due to Heart Failure
Death due to Acute Myocardial Infarction
Sudden Cardiac Death
Death due to Stroke
Death due to other cardiovascular causes
Hospitalization for heart failure
Non-Cardiovascular death
Hospitalization for myocardial infarction
Hospitalization for stroke
Other Cardiovascular hospitalizations
Urgent heart failure visits

Therefore, any event that possibly constitutes one of these endpoints is evaluated by the Clinical Events Classification (CEC) Committee by a procedure to be described in separate documentation.

### Adjudication by the Clinical Endpoint Classification (CEC) Committee

A Clinical Event Committee (CEC) is created for this trial to review and adjudicate each suspected endpoint event while blinded to treatment in this study. The CEC for this trial consists of cardiologists, neurologists, and physicians with clinical expertise from DCRI or other academic institutions. The CEC Chair leads the development of the definitions of endpoints, instructions for interpretation, and provides ongoing oversight to the CEC members for this trial to ensure that events are adjudicated in consistent fashion over time. The CEC members, as well as those overseeing the CEC, are not investigators in the study, or otherwise directly associated with the sponsor, and remain blinded to treatment throughout the study and the adjudication process.

The CEC and the adjudication process are described in detail in a separate CEC charter.

### Adjudicated Endpoints

### Death

All deaths are categorized as Cardiovascular or non-Cardiovascular based on the definitions below. In addition, all deaths are further sub-typed based on the specific cardiovascular categories defined below. Non-cardiovascular deaths are not further adjudicated.

The cause of death was determined by the principal condition that caused the death, not the immediate mode of death. For example, if a participant hospitalized and undergoing treatment for worsening heart failure dies of ventricular tachycardia, this is classified as a heart failure death. CEC physicians utilize all available information provided, along with clinical expertise in their adjudication of cause of death.

### Death Due to Cardiovascular Death

Cardiovascular death includes death resulting from an acute myocardial infarction (MI), sudden cardiac death, death due to heart failure, death due to stroke, and death due to other cardiovascular (CV) causes.

### Death Due to Heart Failure

Death due to heart failure refers to a death in association with clinically worsening symptoms and/or signs of heart failure regardless of heart failure etiology. Deaths due to heart failure can have various etiologies, including single or recurrent myocardial infarctions, ischemic or non-ischemic cardiomyopathy, hypertension, or valvular disease. Deaths that occur during a heart failure hospitalization will generally be attributed to heart failure, even if there is another immediate mode of death (e.g., ventricular fibrillation). Deaths that occur in hospice or other similar palliative care setting for heart failure will generally be attributed to heart failure.

### Death Due to Acute Myocardial Infarction

Death due to acute MI refers to a death by any CV mechanism (e.g. arrhythmia, sudden death, heart failure, stroke, pulmonary embolus, peripheral arterial disease) 30 days after a MI related to the immediate consequences of the MI, such as progressive heart failure or recalcitrant arrhythmia. We note that there may be assessable mechanisms of CV death during this time period, but for simplicity, if the CV death occurs 30 days of the MI, it will be considered a death due to MI.

Acute MI should be verified to the extent possible by the diagnostic criteria outlined for CV hospitalization for acute MI, or by autopsy findings showing recent MI or recent coronary thrombosis. Death resulting from a procedure to treat a MI, percutaneous coronary intervention (PCI), coronary artery bypass graft surgery (CABG), or to treat a complication resulting from MI should also be considered death due to acute MI.

If death occurs before biochemical confirmation of myocardial necrosis can be obtained, adjudication should be based on clinical presentation and ECG evidence. Sudden cardiac death, if accompanied by symptoms suggestive of myocardial ischemia, new ST elevation, new left bundle-branch block (LBBB), or evidence of fresh thrombus by coronary angiography and/or at autopsy should be considered death resulting from an acute myocardial infarction, even if death occurs before blood samples or 12-lead ECG could be obtained, or at a time before the appearance of cardiac biomarkers in the blood. Death resulting from a procedure to treat a myocardial infarction (e.g. PCI, CABG), or to treat a complication resulting from MI, should also be considered death due to acute MI.

### Sudden Cardiac Death

Sudden Cardiac Death refers to death that occurs unexpectedly and not following an acute MI, and includes the following deaths:
Death witnessed and occurring without new or worsening symptoms.
Death witnessed within 60 minutes of the onset of new or worsening cardiac symptoms, unless the symptoms suggest acute myocardial infarction.
Death witnessed and attributed to an identified arrhythmia (e.g., captured on an ECG recording, witnessed on a monitor, or unwitnessed but found on implantable cardioverter-defibrillator (ICD) review)
Death after unsuccessful resuscitation from cardiac arrest. (e.g., ICD unresponsive sudden cardiac death, pulseless electrical activity arrest)
Death after successful resuscitation from cardiac arrest and without identification of a specific cardiac or non-cardiac etiology
Unwitnessed death in a participant seen alive and clinically stable 24 hours prior to being found dead without any evidence supporting a specific non-cardiovascular cause of death (information about the participant's clinical status preceding death should be provided, if available)

### General Considerations

Unless additional information suggests an alternate specific cause of death (e.g. Death due to Other Cardiovascular Causes), if a participant is seen alive 24 hours (or a reasonable period when otherwise clinically stable) of being found dead OR circumstances suggest sudden death, sudden cardiac death should be recorded

Typical scenarios include:
Participant well the previous day but found dead in bed the next day
Participant found dead at home on the couch with the television on

For participants who were not observed alive within 24 hours of death, undetermined cause of death should be recorded (e.g., participant found dead in bed, but who had not been seen by family for several days).

“Undetermined cause of death” is considered as “CV death” for purpose of analysis.

### Death Due to Stroke

Death due to Stroke refers to death after a stroke that is either a direct consequence of the stroke or a complication of the stroke. Acute stroke should be verified to the extent possible by the diagnostic criteria outlined for Hospitalization for Stroke below.

### Death Due to Other Cardiovascular Causes

Death due to Other Cardiovascular Causes refers to a CV death not included in the above categories but with a specific, known cause (e.g., pulmonary embolus or peripheral arterial disease).

### Non-Cardiovascular Death

Non-cardiovascular death is defined as any death that is not thought to be CV in nature. Deaths from Non-CV causes will not be further sub-classified.

### Undetermined Cause of Death

Death not attributable to one of the above categories of CV death, or to a non-CV cause. Inability to classify the cause of death may be due to lack of information (e.g. the only information is “participant died”), or when there is insufficient supporting information or detail to assign the cause of death. In general, most deaths should be classified as CV or non-CV, and the use of this category of death, therefore, should be discouraged and should apply to few participants.

All deaths adjudicated as “undetermined cause” will be presumed cardiovascular deaths, and as such, are part of the cardiovascular mortality endpoint.

### Cardiovascular Hospitalizations

The participant's length-of-stay in hospital extends for at least 24 hours (or a change in calendar date, if admission and discharge times are unavailable).

### Hospitalization for Heart Failure

A Heart Failure hospitalization is defined as an event that meets ALL of the following criteria:
1) The participant is admitted to the hospital with a primary diagnosis of heart failure
2) The participant's length-of-stay in hospital extends for at least 24 hours (or a change in calendar date, if admission and discharge times are unavailable).
3) The participant exhibits documented new or worsening symptoms due to heart failure on presentation, including at least ONE of the following:
a. Dyspnea (dyspnea with exertion, dyspnea at rest, orthopnea, paroxysmal nocturnal dyspnea)
b. Decreased exercise tolerance
c. Fatigue
d. Other symptoms of worsened end-organ perfusion or volume overload (e.g., confusion, somnolence, edema, etc.)
4) The participant has objective evidence of new or worsening heart failure, consisting of at least TWO physical examination findings OR one physical examination finding and at least ONE laboratory criterion), including:
a. Physical examination findings considered to be due to heart failure, including new or worsened:
i. Peripheral edema
ii. Increasing abdominal distention or ascites (in the absence of primary hepatic disease)
iii. Pulmonary rales, crackles, or crepitations
iv. Increased jugular venous pressure and/or hepatojugular reflux
v. S3 gallop
vi. Clinically significant or rapid weight gain thought to be related to fluid retention (usually more than 3-4 lbs in 3-4 days)
b. Laboratory evidence of new or worsening heart failure, if obtained within 24 hours of presentation, including:
i. Increased B-type natriuretic peptide (BNP)/N-terminal pro-BNP (NTproBNP) concentrations consistent with decompensation of heart failure (such as BNP >500 pg/mL or NT-proBNP >2,000 pg/mL). In patients with chronically-elevated natriuretic peptides, a significant (1.25×) increase should be noted above baseline.
ii. Radiological evidence of pulmonary congestion
iii. Non-invasive diagnostic evidence of clinically significant elevated left- or right-sided ventricular filling pressure or low cardiac output. For example, echocardiographic criteria could include: E/e′>15 or D-dominant pulmonary venous inflow pattern, plethoric inferior vena cava with minimal collapse on inspiration, or decreased left ventricular outflow tract (LVOT) minute stroke distance (time velocity integral (TVI)

### OR

iv. Invasive diagnostic evidence with right heart catheterization showing a pulmonary capillary wedge pressure (pulmonary artery occlusion pressure) ≥18 mmHg, central venous pressure ≥12 mmHg, or a cardiac index <2.2 L/min/m2.
5) The participant receives initiation or intensification of treatment specifically for heart failure, including at least ONE of the following:
a. Augmentation in oral diuretic therapy
b. Intravenous diuretic, or vasoactive agent (e.g. positive inotrope, vasopressor or vasodilator)
c. Mechanical or surgical intervention, including mechanical circulatory support (e.g., intra-aortic balloon pump, ventricular assist device, extracorporeal membrane oxygenation, total artificial heart)
d. Mechanical fluid removal (e.g., ultrafiltration, Hemofiltration, dialysis)

### Hospitalization for Myocardial Infarction

Acute MI is adjudicated when a participant demonstrates at least one of the following biochemical indicators of myocardial necrosis:
CK-MB greater than 2×ULN or Troponin I or T greater than 2×ULN, with a typical pattern of rise and fall consistent with MI AND at least one of the two following criteria:
Typical clinical presentation consistent with MI defined as typical cardiac ischemic type pain/discomfort or dyspnea felt to be due to ischemia

### OR

Typical ECG changes consisting of any of the following:
New abnormal Q waves (or new R waves in lead VI-V2) in at least two consecutive leads
Evolving, ischemic ST segment or T wave changes in at least two consecutive leads
New left bundle-branch block (LBBB)

### Hospitalization for Stroke

Stroke is defined as an acute episode of focal or global neurologic dysfunction caused by brain, spinal cord, or retinal vascular injury a result of hemorrhage or infarction. To be classified as a stroke, duration of a focal/global neurological deficit must have a duration >24 hours or imaging confirmation clearly documenting a new hemorrhage or infarct. Events may be classified as a stroke if symptoms were <24 hours due to either pharmacologic or non-pharmacologic interventions or the stroke resulted in death in <24 hours.

### Classification:

Ischemic Stroke: Ischemic stroke is defined as an acute episode of focal cerebral, spinal, or retinal dysfunction caused by infarction of central nervous system tissue hemorrhage may be a consequence of ischemic stroke. In this situation, the stroke is an ischemic stroke with hemorrhagic transformation and not a hemorrhagic stroke.
Hemorrhagic Stroke: Hemorrhagic stroke is defined as an acute episode of focal or global cerebral or Spinal dysfunction caused by intraparenchymal, intraventricular, or subarachnoid hemorrhage.
Undetermined Stroke: Undetermined stroke is defined as an acute episode of focal or global Neurological dysfunction caused by presumed brain, spinal cord, or retinal vascular injury as a result Of hemorrhage or infarction, but with insufficient information to allow categorization as ischemic or hemorrhagic stroke.

### Other Cardiovascular Hospitalizations

Urgent and unscheduled hospitalizations for other cardiovascular causes that do not meet the criteria for the specific events listed above will be classified as hospitalization for other cardiovascular causes. Examples would include hospitalization for cardiac chest pain that does not meet the criteria for MI, hospitalization for arrhythmias, hospitalization for pulmonary embolism, etc. These hospitalizations will not be further sub-classified by the CEC.

### Other Events: Urgent Heart Failure Visit

An urgent heart failure visit is defined as an event that meets all the following:
1) The participant has an urgent, unscheduled office/practice or emergency department visit for a primary diagnosis of heart failure, but not meeting the criteria for a heart failure hospitalization
2) Signs and symptoms that constitute a heart failure hospitalization [i.e., 3) symptoms, 4) physical examination findings/laboratory evidence of new or worsening heart failure, as indicated above] must be met
3) The participant receives initiation or intensification of treatment specifically for heart failure, as detailed in the above section with the exception of oral diuretic therapy, which will not be sufficient.

1-15. (canceled)

16. A method of treating fluid overload, a fluid retention or a fluid accumulation in a patient in need of a treatment with a diuretic, the method comprising:
intravenously administering a dose of ferric carboxymaltose to the patient in need of the diuretic treatment.

17. The method of claim 16 wherein the patient is suffering from heart failure with iron deficiency or the patient is iron deficient and at risk of developing heart failure.

18. The method of claim 17, wherein the heart failure is selected from a chronic or an acute heart failure.

19. The method of claim 17, wherein the heart failure is selected from systolic or diastolic.

20. The method of claim 17, wherein the heart failure is selected from one of a preserved ejection fraction, a mid-range ejection fraction or a reduced ejection fraction.

21. The method of claim 16, wherein the dose of ferric carboxymaltose ranges between 100 mg and 2000 mg of elemental iron.

22. The method of claim 16, wherein the dose of ferric carboxymaltose ranges between 200 mg and 1500 mg of elemental iron.

23. The method of claim 16, wherein a dose of ferric carboxymaltose is selected from 100 mg, 200 mg, 500 mg, 750 mg or 1000 mg of elemental iron.

24. The method of claim 16, wherein the ferric carboxymaltose is administered on a first day of the treatment and after seven days from the first day of the treatment.

25. The method of claim 24, wherein the ferric carboxymaltose is further administered after four weeks, six weeks, twelve weeks, sixteen weeks, twenty-four weeks and/or six months from the first day of the treatment.

26. The method of claim 25, wherein the administration of the ferric carboxymaltose is repeated every six months.

27. The method of claim 16, wherein the patient has an increased relative plasma volume status (PVS) of at least −3.5%.

28. The method of claim 27, wherein, after administering the ferric carboxymaltose, the relative PVS results in a reduction of by at least 1.0% point.

29. A method for treating a congestion, the method comprising:
intravenously administering a dose of ferric carboxymaltose to a patient in need thereof.

30. The method of claim 29, wherein the congestion comprises a fluid overload, a fluid retention or a fluid accumulation.

31. The method of claim 29, wherein the congestion is associated with an impaired function of the heart or associated with deterioration of the function of the heart.

32. The method of claim 29, wherein the patient has a heart failure with iron deficiency, or is iron deficient and at risk of developing the heart failure.

33. The method of claim 31, wherein the patient has a heart failure with iron deficiency, or is iron deficient and at risk of developing the heart failure.

34. The method of claim 31, wherein the impaired function of the heart, or the deterioration of the function of the heart comprises one of a left-ventricular impairment or dysfunction of the heart, a right-ventricular impairment or dysfunction of the heart, or a biventricular impairment or dysfunction of the heart.

35. The method of claim 29, wherein the treating of the congestion comprises one of an improvement, a delay of onset, a delay of recurrence, a delay of deterioration, a prevention of onset, a prevention of recurrence, or a prevention of deterioration of congestion, of at least one of the impaired function of the heart and the deterioration of the heart.
